(19) United States
(12) Patent Application Publication
(10) Pub. No.: US 2026/0092327 A1
(43) Pub. Date: Apr. 2, 2026

(54) METHODS OF MONITORING MUTATIONS IN TREATMENT OF COLORECTAL CANCER

(71) Applicant: Cardiff Oncology, Inc., San Diego, CA (US)

(72) Inventors: Maya Ridinger, San Diego, CA (US); Errin Samuelsz, San Diego, CA (US); Tod Smeal, San Diego, CA (US)

(21) Appl. No.: 19/109,436

(22) PCT Filed: Sep. 8, 2023
(86) PCT No.: PCT/US2023/073693

§ 371 Date: Mar. 6, 2025

Related U.S. Application Data
(60) Provisional application No. 63/404,817, filed on Sep. 8, 2022.

(51) Int. Cl. C12Q 1/6886; A61K 31/198; A61K 31/4412; A61K 31/4425; A61K 31/496; A61K 31/517; A61K 31/519; A61K 31/551; A61K 39/00; A61P 35/00; C07K 16/22

(52) U.S. Cl. CPC C12Q 1/6886; A61K 31/198; A61K 31/4412; A61K 31/4425; A61K 31/496; A61K 31/517; A61K 31/519; A61K 31/551; A61P 35/00; C07K 16/22; A61K 2039/505; C12Q 2600/106; C12Q 2600/118; C12Q 2600/156

(57) ABSTRACT

Provided includes methods, compositions and kits for improving outcome of a treatment for colorectal cancer, and methods, compositions and kits for determining responsiveness of a subject to a treatment for colorectal cancer. Determining responsiveness can comprise determining change(s) in mean level of somatic mutations of at least three genes in the subject. The treatment for colorectal cancer can comprise administering PLK1 inhibitor (e.g., onvansertib) and bevacizumab to the subject.

### RELATED APPLICATIONS

The present application is a U.S. national phase application under 35 U.S.C. § 371 of International Application No. PCT/US2023/073693, filed on Sep. 8, 2023 and published as WO 2024/054951 A1 on Mar. 14, 2024, which claims priority under 35 U.S.C. § 119(e) to U.S. Provisional Application No. 63/404,817, filed Sep. 8, 2022. The content of each of these related applications is incorporated herein by reference in its entirety.

### BACKGROUND

### Field

The present disclosure relates generally to cancer treatment. More specifically, methods for predicting and monitoring effectiveness of a cancer treatment are provided.

### Description of the Related Art

The Polo-like kinase 1 (PLK1) is a serine/threonine kinase and the most well characterized member of this family of 5 closely related regulatory proteins. PLK-1 is a master regulator of mitosis via its control of the entry and progression of cells into and through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 controls kinetochore interactions with the spindle microtubules that is required for successful separation and segregation chromatids to the appropriate mother and daughter cells. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is aberrantly overexpressed in a variety of human cancers and is correlated with cellular proliferation and poor prognosis. There is a need for methods for predicting/determining clinical benefits and outcome for cancer treatments involving PLK1 inhibitors.

### SUMMARY

Disclosed herein include methods for predicting or determining responsiveness of a subject to a treatment for colorectal cancer. The method can comprise: treating the subject with colorectal cancer; detecting change(s) in mean level of somatic mutations of at least three genes in the subject, and determining the responsiveness of the subject to the treatment for colorectal cancer based on the detected change(s) in mean level of somatic mutations. In some embodiments, the treatment for colorectal cancer comprises administering a PLK1 inhibitor and bevacizumab to the subject. In some embodiments, the treatment further comprises a chemotherapy. The at least three genes can comprise KRAS, APC and TP53 genes. The method can, in some embodiments, comprise: treating the subject with colorectal cancer; detecting change(s) in mean level of somatic mutations of KRAS and at least one additional gene in the subject, and determining the responsiveness of the subject to the treatment for colorectal cancer based on the detected change(s) in mean level of somatic mutations. The treatment for colorectal cancer comprises administering a PLK1 inhibitor and bevacizumab to the subject. In some embodiments, the treatment further comprises a chemotherapy. In some embodiments, the at least one additional gene is APC gene or TP53 gene. In some embodiments, the method comprises detecting change(s) in mean level of somatic mutations of KRAS, APC and TP53 in the subject. In some embodiments, the method comprises detecting change(s) in mean level of somatic mutations of KRAS, APC, TP53 and one or more additional genes in the subject.

In some embodiments, the at least three genes further comprise one or more genes selected from AKT1, ALK, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TSC1, and VHL. In some embodiments, the at least three genes further comprise one or more genes selected from AR, BRAF, CCND1, CCND2, CCNE1, COK4, COK6, EGFR, ERBB2, FGFR1, FGFR2, KIT, MET, MYC, POGFRA, PIK3CA, and RAF1. In some embodiments, the at least three genes further comprise one or more genes selected from ALK, FGFR2, FGFR3, NTRK1, RET, and ROS1. In some embodiments, the at least three genes further comprise one or more genes selected from the group consisting of APC, AR, ATM, BRAF, BRCA1, BRCA2, CDK4, CDK6, CDK12, EGFR, ERBB2, HRAS, KIT, KRAS, MAPK1, MAPK3, MET, MYC, NRAS, PIK3CA, PTEN, RB1, STK11, and TP53.

The somatic mutation can be, for example, a substitution, an insertion, a deletion, an amplification, a fusion or a combination thereof. In some embodiments, the somatic mutation is a point mutation, e.g., a single-nucleotide variant (SNV). In some embodiments, the somatic mutation is an insertion-deletion mutation (indel). As described herein, detecting change(s) in mean level of somatic mutations of at least three genes includes the situations in which one or two of the at least three genes are not mutated in the subject, and thus the mean level is calculated using only the two or one of the at least three genes that are mutated? For example, for a patient having mutations in KRAS and TP53 genes but no APC variants at baseline, the mean level is calculated from KRAS and TP53 MAFs, unless APC mutation(s) is detected in the subsequent timepoint for the subject.

In some embodiments, detecting change(s) in the mean level of somatic mutations of the at least three genes in the subject comprises detecting one or more somatic mutations of the at least three genes in the subject (1) during the subject is treated for cancer, (2) before the subject is treated for cancer, (3) after the subject is treated for cancer, or a combination thereof. In some embodiments, detecting change(s) in the mean level of somatic mutations of the at least three genes in the subject comprises detecting one or more somatic mutations of the at least three genes two or more times in the subject. In some embodiments, at least two of the two or more times occur within 5, 7, 14, 28, 35 or 42 days. In some embodiments, change(s) in the mean level of somatic mutations of the at least three genes comprises (1) change(s) in the mean level of somatic mutations of the at least three genes during the subject is treated for cancer, (2) change(s) in the mean level of somatic mutations of the at least three genes from before the subject is treated for cancer to during the subject is treated for cancer, or a combination thereof.

In some embodiments, detecting change(s) in the mean level of somatic mutations of at least three genes comprises detecting mean variant allele frequency (VAF) of the at least three genes. In some embodiments, the mean variant allele frequency (VAF) is mean mutant allelic frequency (MAF). In some embodiments, change(s) in the mean level of somatic mutations of at least three genes is a ratio of mean MAF of at least one additional time point to a first time point. In some embodiments, the first time point is earlier than the at least one additional time point.

In some embodiments, detecting change(s) in the mean level of somatic mutations of the at least three genes in the subject comprises detecting change(s) in the mean level of somatic mutations of the at least three genes in a biological sample from the subject, or derivative thereof. In some embodiments, the biological sample comprises a bodily fluid, whole blood, plasma, one or more tissues, one or more cells, or a combination thereof. The bodily fluid can be or can comprise blood, plasma, urine, or a combination thereof. In some embodiments, the bodily fluid is blood.

The biological sample can comprise circulating tumor DNA (ctDNA), cell-free DNA (cfDNA), circulating tumor cell (CTC), or a combination thereof. In some embodiments, the biological sample comprises ctDNA. The method can comprise analyzing the ctDNA using polymerase chain reaction (PCR) or next generation sequencing (NGS). The method can comprise analyzing the ctDNA using NGS, e.g., a high-throughput NGS.

In some embodiments, the subject has one or more somatic mutations in the at least three genes before being treated with the PLK1 inhibitor. In some embodiments, the subject does not have one or more somatic mutations in the at least three genes before being treated with the PLK1 inhibitor. In some embodiments, detecting change(s) in the mean level of the somatic mutation(s) of the at least three genes in the subject comprises detecting one or more somatic mutations of the at least three genes emerged in the subject after the subject being treated with the PLK1 inhibitor and bevacizumab.

In some embodiments, determining the responsiveness of the subject comprises determining if the subject is a responder of the treatment, if the subject is or is going to be in complete recover (CR), or if the subject is or is going to be in partial remission (PR).

In some embodiments, the change in the mean level of the somatic mutation(s) of the at least three genes in the responder is a decrease of at least 50%, at least 75%, at least 90%, at least 95%, or at least 99%. In some embodiments, the change in the mean level of the somatic mutations of the at least three genes in the responder is a decrease of at least 99%. In some embodiments, the change in the mean level of the somatic mutations of the at least three genes identifies the responders more accurately relative to change(s) in mutation(s) of one gene. In some embodiments, the one gene is KRAS. In some embodiments, a specificity and/or an accuracy of identifying responders using the change in the mean level of the somatic mutations of the at least three genes is at least 3%, at least 5%, at least 7%, at least 10%, at least 15%, or at least 20% higher, relative to using change(s) in mutation(s) of one gene. In some embodiments, the one gene is KRAS.

In some embodiments, determining the responsiveness of the subject can comprise determining progression-free survival (PFS) of the subject. In some embodiments, determining the responsiveness of the subject comprises determining overall survival (OS) of the subject. In some embodiments, determining the responsiveness of the subject comprises determining if the subject has a partial response to the treatment, if the subject has a complete response to the treatment, if the subject has a stable disease (SD) status, or if the subject has a progressive disease (PD) status.

In some embodiments, the cancer treatment with the PLK1 inhibitor and bevacizumab is maintained if the change in mean MAF of the at least three genes is a decrease of at least 25%, at least 50%, or at least 75%. In some embodiments, the decrease is at least 99%. In some embodiments, the decrease is detected at the end of cycle 1 of the cancer treatment or at day 1 of cycle 2 of the cancer treatment. In some embodiments, the cancer treatment is for at least one week, at least two weeks, at least three weeks, at least one month, at least three months, or at least six months. In some embodiments, the cancer treatment with the PLK1 inhibitor and bevacizumab is modified or discontinued if the change in mean MAF the at least three genes is a decrease of less than 50%, less than 25%, or less than 10%. In some embodiments, the decrease is detected at the end of cycle 1 of the cancer treatment or at day 1 of cycle 2 of the cancer treatment. In some embodiments, the cancer treatment with the PLK1 inhibitor and bevacizumab is maintained if mean level of mutations in the at least three genes in the samples decreases to below 0.01% or below 0.001% of mean level of the at least three genes in the sample.

Disclosed herein include methods of improving outcome of a treatment for colorectal cancer. The method comprises: detecting mean variant allele frequency of a first set of at least three genes in a subject at a first time point in a first sample; detecting mean variant allele frequency of a second set of at least three gene in the subject at one or more additional time points in one or more additional samples; determining the difference of the mean variant allele frequency between the first and the one or more additional samples; and continuing the treatment for colorectal cancer to the subject if the subject is indicated as responsive to the treatment for colorectal cancer, or discontinuing the treatment for colorectal cancer to the subject and/or starting a different treatment to the subject if the subject is not indicated as responsive to the treatment for colorectal cancer. In some embodiments, the first set of the at least three genes and/or the second set of the at least three genes comprise KRAS, APC and TP53 genes. In some embodiments, the first time point is before the subject starts the treatment for colorectal cancer, or during the treatment for colorectal cancer. In some embodiments, the treatment for colorectal cancer comprises administering a PLK1 inhibitor and bevacizumab to the subject. In some embodiments, the at least one of the one or more additional time points is during the treatment for colorectal cancer. In some embodiments, a decrease in the mean variant allele frequency in at least one of the one or more additional samples relative to the first sample indicates the subject as responsive to the treatment for colorectal cancer.

In some embodiments, the first time point is before the subject starts the treatment for colorectal cancer. In some embodiments, at least two of the additional time points are during the treatment for colorectal cancer.

Disclosed herein include methods of treating colorectal cancer. The method comprises: treating a subject with colorectal cancer; determining a decrease, relative to a mean variant allele frequency of a first set of at least three genes in a first sample of the subject obtained at a first time point before the subject receives the treatment for colorectal cancer or during the treatment for colorectal cancer, in a mean variant allele frequency of a second set of at least three genes in a second sample of the subject obtained at a second time point after the subject starts receiving the treatment for colorectal cancer; and continuing with the treatment for colorectal cancer. In some embodiments, the treating comprises administering a PLK1 inhibitor and bevacizumab to the subject. In some embodiments, the first set of at least three genes and the second set of at least three genes comprise KRAS, APC and TP53 genes.

In some embodiments, the first set of at least three genes detected at the first time point are the same as the second set of at least three genes detected at the second time point. In some embodiments, the first set of at least three genes detected at the first time point are different from the second set of at least three genes detected at the second time point.

In some embodiments, the first set of at least three genes and/or the second set of at least three genes further comprise one or more genes selected from AKT1, ALK, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TSC1, and VHL. In some embodiments, the first set of at least three genes and/or the second set of at least three genes further comprise one or more genes selected from AR, BRAF, CCND1, CCND2, CCNE1, COK4, COK6, EGFR, ERBB2, FGFR1, FGFR2, KIT, MET, MYC, POGFRA, PIK3CA, and RAF1. In some embodiments, the first set of at least three genes and/or the second set of at least three genes further comprise one or more genes selected from ALK, FGFR2, FGFR3, NTRK1, RET, and ROS1. In some embodiments, the first set of at least three genes and/or the second set of at least three genes further comprise one or more genes selected from APC, AR, ATM, BRAF, BRCA1, BRCA2, CDK4, CDK6, CDK12, EGFR, ERBB2, HRAS, KIT, KRAS, MAPK1, MAPK3, MET, MYC, NRAS, PIK3CA, PTEN, RB1, STK11, and TP53.

In some embodiments, the first set of at least three genes and/or the second set of at least three genes have somatic mutation(s). In some embodiments, the somatic mutation is a substitution, an insertion, a deletion, an amplification, a fusion or a combination thereof. The somatic mutation can be, for example, a point mutation and/or an insertion-deletion mutation (indel). In some embodiments, the point mutation is a single-nucleotide variant (SNV).

In some embodiments, the somatic mutation(s) in the first set of at least three genes detected at the first time point are the same as the somatic mutation(s) in the second set of at least three genes detected at the second time point. In some embodiments, the somatic mutation(s) in the first set of at least three genes detected at the first time point are different from the somatic mutation(s) in the second set of at least three genes detected at the second time point.

In some embodiments, the first time point is prior or immediately prior to the cancer treatment. In some embodiments, the first time point is during the cancer treatment. In some embodiments, the first time point is at day 5, 7, 14, or 28 of the cancer treatment. In some embodiments, the one or more additional time points are during the cancer treatment. In some embodiments, the one or more additional time points are on day 5, 7, 14, 28, 35 or 42 of the cancer treatment. In some embodiments, the first time point and at least one of the one or more additional time points are during the first cycle of the cancer treatment. In some embodiments, at least one of the one or more additional time points are during the first cycle of the cancer treatment, and at least one of the one or more additional time points are during the second cycle of the cancer treatment. In some embodiments, the mean variant allele frequency is mean mutant allelic frequency (MAF).

In some embodiments, the detecting step comprises detecting mean variant allele frequency in the first set of at least three genes and/or the second set of at least three genes in a biological sample from the subject, or derivative thereof. The biological sample can be or can comprise a bodily fluid, whole blood, plasma, one or more tissues, one or more cells, or a combination thereof. The bodily fluid can be or can comprise blood, plasma, urine, or a combination thereof.

In some embodiments, the biological sample comprises circulating tumor DNA (ctDNA), circulating tumor cell (CTC), or a combination thereof. The method can comprise analyzing the ctDNA using polymerase chain reaction (PCR) or next generation sequencing (NGS). In some embodiments, the NGS is a high-throughput NGS.

In some embodiments, the subject has one or more mutations in the first set of at least three genes and/or the second set of at least three genes before being treated with the PLK1 inhibitor and bevacizumab. In some embodiments, the subject does not have mutations in the first set of at least three genes and/or the second set of at least three genes before being treated with the PLK1 inhibitor and bevacizumab. In some embodiments, the mean variant allele frequency of a second set of at least three gene decreases to below 0.01% or below 0.001% of mean level of the at least three genes in a sample.

In some embodiments, the subject has received one or more prior cancer treatment. In some embodiments, the colorectal cancer is metastatic colorectal cancer. In some embodiments, the PLK1 inhibitor is onvansertib, BI2536, volasertib (BI 6727), GSK461364, HMN-176, HMN-214, AZD1775, CYC140, rigosertib (ON-01910), MLN0905, TKM-080301, TAK-960, Ro3280, or a combination thereof. In some embodiments, the PLK1 inhibitor is onvansertib.

In some embodiments, the treatment for colorectal cancer comprises administration of onvansertib every day in a cycle of 28 days. In some embodiments, the treatment for colorectal cancer comprises administration of onvansertib for the first 21 days and not the last 7 days in a cycle of 28 days. In some embodiments, the treatment for colorectal cancer comprises administration of onvansertib for ten days in a cycle of 28 days. In some embodiments, the treatment for colorectal cancer comprises administration of onvansertib for five days in the first 14 days and five days in the second 14 days in a cycle of 28 days. In some embodiments, the treatment for colorectal cancer comprises administration of onvansertib on days 1-5 and days 15-19 in a cycle of 28 days.

In some embodiments, the treatment comprises administration of onvansertib at 6 mg/m²-24 mg/m², for example at 6 mg/m²-12 mg/m² or at 12 mg/m²-18 mg/m². In some embodiments, the treatment comprises administration of onvansertib at 12 mg/m² or 15 mg/m². In some embodiments, a maximum concentration (Cₘₐₓ) of onvansertib in a blood of the subject is from about 100 nmol/L to about 1500 nmol/L. In some embodiments, an area under curve (AUC) of a plot of a concentration of onvansertib in a blood of the subject over time is from about 1000 nmol/L·hour to about 400000 nmol/L·hour. In some embodiments, a time (Tmax) to reach a maximum concentration of onvansertib in a blood of the subject is from about 1 hour to about 5 hours. In some embodiments, an elimination half-life (T_{1/2}) of onvansertib in a blood of the subject is from about 10 hours to about 60 hours.

The treatment for colorectal cancer can further comprise administering to the subject at least one additional cancer therapeutics or cancer therapy. In some embodiment, the additional cancer therapeutics or cancer therapy can be, or can comprise, FOLFIRI, FOLFOX, XELOX (CAPOX), or FOLFOXTRI. In some embodiments, the additional cancer therapeutics or cancer therapy comprises FOLFIRI. In some embodiments, the PLK inhibitor and the additional cancer therapeutics or cancer therapy are co-administered simultaneously or sequentially.

In some embodiments, the cancer treatment comprises one or more cycles, and change(s) in the mean level of somatic mutations of the at least three genes or mean variant allele frequency of the first set of at least three genes and/or the second set of at least three genes is detected before, during and/or after each cycle of the cancer treatment. In some embodiments, each cycle of treatment is at least 21 days. In some embodiments, each cycle of treatment is from about 21 days to about 28 days. In some embodiments, each cycle of treatment is 28 days. In some embodiments, the subject is human.

Disclosed herein include uses of a PLK1 inhibitor and bevacizumab as a treatment of a subject with colorectal cancer. In some embodiments, the responsiveness of the subject to the treatment for colorectal cancer is determined using a method disclosed herein. Disclosed herein include uses of a PLK1 inhibitor and bevacizumab as a treatment of a subject with colorectal cancer. In some embodiments, the treatment outcome is improved using a method disclosed herein. Disclosed herein include uses of a PLK1 inhibitor and bevacizumab as a treatment of a subject with colorectal cancer. In some embodiments, the subject is treated using a method disclosed herein. In some embodiments, the PLK1 inhibitor is onvansertib.

### BRIEF DESCRIPTION OF THE DRAWINGS

FIG. 1 depicts non-limiting exemplary embodiments and data related to workflow of ctDNA analysis.

FIG. 2 depicts non-limiting exemplary embodiments and data related to workflow of molecular response test.

FIG. 3A-FIG. 3B depict non-limiting exemplary embodiments and data related to a comparison of molecular response with KRAS-only response within the context of next-generation sequencing (NGS). FIG. 3A depicts a comparison of Delta Mutation Allelic Frequencies (MAF) between molecular response and KRAS-only response. FIG. 3B depicts the correlation of Delta MAF of molecular response and KRAS-only response.

FIG. 4 depicts non-limiting exemplary embodiments and data related to ROC Curve for clinical response prediction.

FIG. 5 depicts non-limiting exemplary embodiments and data related to KRAS mutations and clinical response.

FIG. 6 depicts non-limiting exemplary embodiments and data related to changes in KRAS MAF and best response.

FIG. 7 depicts non-limiting exemplary embodiments and data related to progression-free survival (PFS) of KRAS responders and non-responders.

FIG. 8 depicts non-limiting exemplary embodiments and data related to the correlation of 3-gene molecular response and 74-gene molecular response

FIG. 9A-FIG. 9B depict non-limiting exemplary embodiments and data related to performance of 3-gene molecular response (FIG. 9B) and 74-gene molecular response (FIG. 9A) using threshold of −90%. In FIG. 9A-FIG. 9B, TP (true positive), FP (false positive), FN (false negative), TN (true negative), PPV (positive predictive value), and NPV (negative predictive value) are shown.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients.

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” or “prevention,” refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response,” “partial remission,” or “PR” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone Marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response,” “complete remission,” “complete recovery,” or “CR” refers to a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. However, a “complete response” does not necessarily indicate that the cancer has been cured. A complete response may be followed by a relapse. A complete response of a target lesion includes disappearance of all target lesions and any pathological lymph nodes (whether target or non-target) having reduction in short axis to <10 mm. A complete response of a non-target lesion includes disappearance of all non-target lesions and normalization of tumor marker level (all lymph nodes must be non-pathological in size (<10 mm short axis)). If tumor markers are initially above the upper normal limit, they need to normalize for a patient to be considered in complete clinical response of a nontarget lesion. The duration of overall CR is measured from the time measurement criteria are first met for CR until the first date that progressive disease is objectively documented, or death due to any cause. Participants without events reported are censored at the last disease evaluation.

As used herein, the term “stable disease” or “SD” means neither sufficient shrinkage to qualify for PR nor sufficient increase to qualify for progressive disease (PD), taking as reference the smallest sum diameters while on study. Duration of stable disease is measured from the start of the treatment until the criteria for progression are met, taking as reference the smallest measurements recorded since the treatment started, including the baseline measurements.

As used herein, the term “progressive disease” or “PD” when refers to a target lesion means at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). When progressive disease or PD refers to a non-target lesion, it means the appearance of one or more new lesions and/or unequivocal progression of existing non-target lesions. Unequivocal progression should not normally trump target lesion status. It must be representative of overall disease status change, not a single lesion increase.

As used herein, the term “best overall response” means the best response recorded from the start of the treatment until disease progression/recurrence (taking as reference for progressive disease the smallest measurements recorded since the treatment started). The patient's best response assignment depends on the achievement of both measurement and confirmation criteria. The duration of an overall response is measured from the time measurement criteria are met for CR or PR (whichever is first recorded) until the first date that recurrent or progressive disease is objectively documented (taking as reference for progressive disease the smallest measurements recorded since the treatment started, or death due to any cause. Participants without events reported are censored at the last disease evaluation).

As used herein, the term “MTD” means maximum tolerated dose.

As used herein, the term “DLT rate” means dose-limiting toxicity rate.

As used herein, the term “IC₅₀” means inhibitory drug concentration that produces 50% of the maximal effect.

As used herein, the term “SEM” means standard error of mean.

As used herein, the term “AUC(x-y)” means area under the curve, wherein “x” is the starting time in hours and “y” is the ending time in hours.

As used herein, the term “C_{avg}” means average concentration. As used herein, the term “Cₘₐₓ” means maximum concentration.

As used herein, the term “ctDNA” means circulating tumor DNA.

As used herein, the term “CXD1” means Day 1 of Cycle X. For example, C1D1 means Day 1 of Cycle 1, which is before the subject receiving the intended treatment. C2D1 means Day 1 of Cycle 2.

As used herein, the term “PK” means pharmacokinetic.

As used herein, the term “TNM” means tumor, lymph nodes, metastasis.

As used herein, the term “concomitant medication” means medications (or treatments), other than the drugs used in a study, are taken or received by the patient during the course of the study (after the administration of the first dose of study drug and before the final study visit assessment).

As used herein, the term “tolerable” means a dose level where ≤1/6 participants have experienced a DLT, or the dose level that is declared the RP2D.

As used herein, the term “adverse event” or “AE” means an untoward medical occurrence in a subject administered a medicinal product that does not necessarily have a causal relationship with this treatment. An AE can be an unfavorable and unintended sign (including an abnormal laboratory finding), symptom, or disease temporally associated with the use of an investigational product, whether or not related to the investigational medicinal product. An adverse event may include worsening or exacerbation of the disease under study; worsening or exacerbation of pre-existing conditions or events; intercurrent illnesses; or drug interactions. Anticipated fluctuations of pre-existing conditions that do not represent a clinically significant exacerbation or worsening are not considered AEs. Surgical procedures are not adverse events; they are therapeutic measures for conditions that require surgery. The condition, provided it develops or is a worsening of a pre-existing condition for which the surgery is required, is the AE. Disease progression is an efficacy endpoint and is not an AE. A clinical event in the setting of disease progression would be considered an AE if it could not be unequivocally attributed to or consistent with expected disease progression.

As used herein, the term “expected adverse event” means an adverse event that are listed or characterized in the current adverse event list, the Package Insert (P.I.), the Investigator Brochure (I.B.) or is included in the informed consent document as a potential risk.

As used herein, the term “unexpected adverse event” means an adverse event that is not listed in the P.I. or current I.B. or not identified. This includes adverse events for which the specificity or severity is not consistent with the description in the P.I. or I.B. For example, under this definition, hepatic necrosis would be unexpected.

As used herein, the term “severe adverse event” or “SAE” means an AE that (1) results in death (i.e., the AE actually causes or leads to death); (2) is life threatening (i.e., the AE, in the view of the investigator, places the subject at immediate risk of death, but does not include an AE that, had it occurred in a more severe form, might have caused death); (3) requires or prolongs inpatient hospitalization; (4) results in persistent or significant disability/incapacity (i.e., the AE results in substantial disruption of the subject's ability to conduct normal life functions); or (5) results in a congenital anomaly/birth defect in a neonate/infant born to a mother exposed to the IMP.

As used herein, the term “definite AE” means the AE is clearly related to the study treatment.

As used herein, the term “probable AE” means the AE is likely related to the study treatment.

As used herein, the term “possible AE” means the AE may be related to the study treatment.

As used herein, the term “unlikely AE” means the AE is doubtfully related to the study treatment.

As used herein, the term “unrelated AE” means the AE is clearly not related to the study treatment.

As used herein, the term “expected disease progression” means an event that is unequivocally related to disease progression, and that the clinical course is consistent with what would be expected for the patient's disease.

As used herein, the term “measurable lesion” means a lesion that can be accurately measured in at least one dimension (longest diameter to be recorded) as ≥20 mm by chest x-ray or ≥10 mm with CT scan, MRI, or calipers by clinical exam. Tumor lesions that are situated in a previously irradiated area might or might not be considered measurable. Cystic lesion thought to represent cystic metastases are measurable lesions if they meet the definition of measurability described above. However, they are target lesions if non-cystic lesions are also present in the same participant. Clinical lesions are measurable when they are superficial (e.g., skin nodules and palpable lymph nodes) and ≥10 mm in diameter as assessed using calipers (e.g., skin nodules).

As used herein, the term “malignant lymph node” means a pathologically enlarged and measurable lymph node with ≥15 mm in short axis when assessed by CT scan.

As used herein, the term “non-measurable disease” means a small lesion (or a site of disease) where the longest diameter <10 mm or pathological lymph nodes with ≥10 to <15 mm short axis. Bone lesions, leptomeningeal disease, ascites, pleural/pericardial effusions, lymphangitis cutis/pulmonitis, inflammatory breast disease, abdominal masses (not followed by CT or MRI), and cystic lesions are examples of non-measurable disease. Cystic lesions that meet the criteria for radiographically defined simple cysts are not malignant lesions (neither measurable nor non-measurable) and are simple cysts.

As used herein, the term “overall survival” or “OS” means the time from randomization (or registration) to death due to any cause. Participants survived are censored on date last known alive.

As used herein, the term “time to progression” or “TTP” means the time from randomization (or registration) to progression. Participants without progression reported are censored on date of last disease evaluation.

As used herein, the term “progression-free survival” or “PFS” means the time from randomization (or registration) to the earlier of progression or death due to any cause. Participants alive without disease progression are censored on date of last disease evaluation.

As used herein, the term “clonal haematopoiesis of indeterminate potential” or “CHIP” means mosaic chromosomal alterations (mCAs), single-nucleotide variants (SNVs) and indels in genes associated with myeloid malignancies, and putative incidental mutations/genomic drift, in the absence of a haematological malignancy. The CHIP mutations can be present at ≥2% variant allele fraction (VAF). Clonal haematopoiesis (CH) is a common, age-related expansion of blood cells with somatic mutations that is associated with an increased risk of haematological malignancies.

As used herein, the term “sensitivity” refers to a measure of the ability of a test to correctly identify an affected individual, or an individual who will develop the trait of interest. The closer the sensitivity is to one, the more accurate the test is in identifying affected individuals. Specifically, the sensitivity refers to the proportion of affected individuals who are correctly diagnosed as such by the test, and is calculated as the number of individuals correctly identified as affected (TP) divided by the total number of affected individuals (TP+FN). A high sensitivity is preferred so that most affected individuals are identified as such by the genetic test. Thus, for a given risk cutoff value, the sensitivity is calculated as the proportion of case individuals with a score higher than the risk cutoff value.

As used herein, the term “specificity” refers to a measure of the ability of a test to correctly identify an unaffected individual, or an individual who will not develop the trait of interest. The closer the specificity is to one, the more accurate the test is in identifying unaffected individuals. Specifically, the specificity refers to the proportion of unaffected individuals who are correctly identified as such by the test, and is calculated as the number of individuals correctly identified as unaffected (TN) divided by the total number of unaffected individuals (TN+FP). A high specificity is preferred so that the number of individuals who are incorrectly identified as affected is minimized. Thus, for a given risk cutoff value, the specificity is calculated as the proportion of control individuals with a score lower than or equal to the risk cutoff value (or, one minus the proportion of control individuals with a score higher than the risk cutoff value).

As used herein, the term “positive predictive value” or “PPV” assesses the reliability of a positive test outcome/result, and is computed as the proportion of people with a positive test result who actually have the trait of interest. In other words, it is the probability that a positive test result accurately identifies an individual who has the trait, and is calculated as the number of individuals correctly identified as affected (TP) divided by the total number of individuals identified as affected by the genetic test (TP+FP). In many cases, a high PPV is preferred so that most individuals who are identified as affected are actually affected. For example, a PPV of 0.98 means that an individual with a positive test result has a 98% chance of having or developing the trait. Thus, for a given risk cutoff value, the PPV can be calculated as the proportion of all individuals with a score higher than the risk cutoff value that are actually in the case group.

As used herein, the term “negative predictive value” or “NPV” assesses the reliability of a negative test outcome/result, and is computed as the proportion of people with a negative test result who do not have the trait of interest. Put another way, it is the probability that a negative test result accurately identifies an individual who does not have the trait, and is calculated as the number of individuals correctly identified as unaffected (TN) divided by the total number of individuals identified as unaffected (TN+FN). A high NPV is sometimes preferred so that most individuals who are identified as unaffected are actually unaffected (e.g., in excluding subjects at risk for adverse events associated with the administration of a specific drug). For example, an NPV of 0.999 means that an individual with a negative test result has only a 0.1% chance of having or developing the trait (e.g., of experiencing the adverse event in response to the drug). Thus, for a given risk cutoff value, the NPV can be calculated as the proportion of all individuals with a score lower than or equal to the risk cutoff value that are actually in the control group.

As used herein, the term “accuracy” refers to a measurement of the overall agreement between the test results and the actual disease state. Accuracy is calculated as the sum of the true positives and true negatives divided by the total number of sample results ((TP+TN)/(TP+TN+FP+FN)). The accuracy of a genetic test may be used to determine which of a set of risk cutoff values may be a useful threshold value in a polygenic test.

Disclosed herein include methods, compositions and kits for determining responsiveness of a subject to a treatment for colorectal cancer, methods, compositions and kits for improving outcome of a treatment for colorectal cancer, and methods, compositions and kits for treating colorectal cancer.

Disclosed herein include methods for predicting or determining responsiveness of a subject to a treatment for colorectal cancer. In some embodiments, the method comprises: treating the subject with colorectal cancer; detecting change(s) in mean level of somatic mutations of at least three genes in the subject, and determining the responsiveness of the subject to the treatment for colorectal cancer based on the detected mean change(s) in somatic mutations. In some embodiments, the treatment for colorectal cancer comprises administering a PLK1 inhibitor and bevacizumab to the subject. In some embodiments, the at least three genes comprise KRAS, APC and TP53 genes.

Disclosed herein include methods of improving outcome of a treatment for colorectal cancer. The method comprises: detecting mean variant allele frequency of a first set of at least three genes in a subject at a first time point in a first sample; detecting mean variant allele frequency of a second set of at least three gene in the subject at one or more additional time points in one or more additional samples; determining the difference of the mean variant allele frequency between the first and the one or more additional samples; and continuing the treatment for colorectal cancer to the subject if the subject is indicated as responsive to the treatment for colorectal cancer, or discontinuing the treatment for colorectal cancer to the subject and/or starting a different treatment to the subject if the subject is not indicated as responsive to the treatment for colorectal cancer. In some embodiments, the first set of the at least three genes and/or the second set of the at least three genes comprise KRAS, APC and TP53 genes. In some embodiments, the first time point is before the subject starts the treatment for colorectal cancer, or during the treatment for colorectal cancer. In some embodiments, the treatment for colorectal cancer comprises administering a PLK1 inhibitor and bevacizumab to the subject. In some embodiments, the at least one of the one or more additional time points is during the treatment for colorectal cancer. In some embodiments, a decrease in the mean variant allele frequency in at least one of the one or more additional samples relative to the first sample indicates the subject as responsive to the treatment for colorectal cancer.

Disclosed herein include methods of treating colorectal cancer. The method comprises: treating a subject with colorectal cancer; determining a decrease, relative to a mean variant allele frequency of a first set of at least three genes in a first sample of the subject obtained at a first time point before the subject receives the treatment for colorectal cancer or during the treatment for colorectal cancer, in a mean variant allele frequency of a second set of at least three gene in a second sample of the subject obtained at a second time point after the subject starts receiving the treatment for colorectal cancer; and continuing with the treatment for colorectal cancer. In some embodiments, the treating comprises administering a PLK1 inhibitor and bevacizumab to the subject. In some embodiments, the first set of at least three genes and the second set of at least three genes comprise KRAS, APC and TP53 genes.

### Cancer

The methods, compositions and kits disclosed herein can be used to predict and determine responsiveness of a subject to a treatment, improve outcome of a treatment of cancer and/or tumor, and treat the cancer and/or tumor. The cancer and/or tumor can be a solid tumor, a liquid tumor, or a combination thereof. In some embodiments, the cancer is colorectal cancer, for example, metastatic colorectal cancer and/or colon cancer.

The cancer can be an invasive cancer unresectable locally advanced or metastatic disease, for example metastatic colorectal cancer (mCRC). In some embodiments, the cancer is an inflammatory cancer. In some embodiment, the cancer has a histological or cytological profile with ER≤10%, PR≤10%, Her-2-neu negative per ASCO/CAP 2018 guidelines (0-1+ by immuno-histochemistry (IHC) or fluorescence in situ hybridization (FISH)-negative).

The cancer and/or tumor can be a cancer and/or tumor having abnormal alterations to PLK1 gene or protein. For example, the abnormal alterations can include one or more PLK1 alterations and/or PLK1 aberrant activation such as copy number alteration (CNA), single-nucleotide variation (SNV), and gene rearrangement or fusions.

The cancer can be a KRAS mutation cancer, for example a KRAS mutation colorectal cancer, e.g., metastatic colorectal cancer. In some embodiments, the KRAS gene mutation(s) comprise mutations at codon 12, codon 13, codon 18, codon 61, codon 117, codon 146, or a combination thereof. In some embodiments, the KRAS gene mutation(s) comprise mutations at codon 12 and/or codon 13. In some embodiments, the KRAS gene mutation(s) comprise G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

Colorectal cancer (CRC) also known as bowel cancer, is a cancer from uncontrolled cell growth in the colon or rectum (parts of the large intestine), or in the appendix and is one of the most frequently diagnosed malignancies and a leading cause of cancer-related deaths worldwide. High degree of mortality associated with CRC is largely due to late disease detection and lack of availability of adequate prognostic biomarkers, including the currently used tumor-node-metastasis (TNM) classification system from the American Joint Committee on Cancer for predicting tumor prognosis and recurrence. In some embodiments, the colorectal cancer is colon cancer, rectal cancer, or metastatic colorectal cancer.

The most common staging system is the TNM (for tumors/nodes/metastases) system, from the American Joint Committee on Cancer (AJCC). The TNM system assigns a number based on three categories. “T” denotes the degree of invasion of the intestinal wall, “N” the degree of lymphatic node involvement, and “M” the degree of metastasis. The broader stage of a cancer is usually quoted as a number I, II, III, IV derived from the TNM value grouped by prognosis; a higher number indicates a more advanced cancer and likely a worse outcome. Details of this system are in the graph below. Colorectal tumors can also be categorized into several stages according to Dukes' stages A-D. Early-stage tumors (Dukes' stages A and B) are generally associated with a relatively favorable outcome, while later stage tumors, presenting with metastasis (Dukes' stage C and D) have poor survival rates. Dukes' stage A, B, C and D corresponds to TNM stage I, II, III and IV, respectively.

TABLE 1
TNM STAGES OF COLORECTAL CANCER

| AJCC stage | TNM stage | TNM stage criteria for colorectal cancer |
|---|---|---|
| 0 | Tis N0 M0 | Tis: Tumor confined to mucosa; cancer-in-situ |
| I | T1 N0 M0 | T1: Tumor invades submucosa |
| I | T2 N0 M0 | T2: Tumor invades muscularis propria |
| II-A | T3 N0 M0 | T3: Tumor invades subserosa or beyond (without other |
| | | organs involved) |
| II-B | T4 N0 M0 | T4: Tumor invades adjacent organs or perforates the |
| | | visceral peritoneum |
| III-1 | T1-2 N1 M0 | N1: Metastasis to 1 to 3 regional lymph nodes. T1 or T2. |
| III-B | T3-4 N1 M0 | N1: Metastasis to 1 to 3 regional lymph nodes. T3 or T4. |
| III-C | any T, N2 M0 | N2: Metastasis to 4 or more regional lymph nodes. Any T. |
| IV | any T, any N, M1 | M1: Distant metastases present. Any T, any N. |

For patients with localized and/or early colorectal cancer, the preferred treatment is complete surgical removal with adequate margins, with the attempt of achieving a cure. Sometimes chemotherapy is used before surgery to shrink the cancer before attempting to remove it (neoadjuvant therapy). The two most common sites of recurrence of colorectal cancer are in the liver and lungs.

In both cancer of the colon and rectum, chemotherapy can be used in addition to surgery in certain cases. In rectal cancer, chemotherapy can be used in the neoadjuvant setting. In certain embodiments, there can be a decision regarding the therapeutic treatment based on biomarker expression. Chemotherapy based on antimetabolites or thymidylate synthase inhibitors such as 5-fluorouracil (5-FU) have been the main treatment for metastatic colorectal cancer. Major progress has been made by the introduction of regimens containing additional cytotoxic drugs, such as irinotecan or oxaliplatin. The combinations commonly used, e.g., irinotecan, fluorouracil, and leucovorin (FOLFIRI); and oxaliplatin, fluorouracil, and leucovorin (FOLFOX) can reach an objective response rate of about 50%. However, these combinations remain inactive in one half of the patients and, in addition, resistance to treatment appear in almost all patients who were initially responders. More recently, two monoclonal antibodies targeting vascular endothelial growth factor Avastin® (bevacizumab) (Genentech Inc., South San Francisco California) and epidermal growth factor receptor Erbitux® (cetuximab) (Imclone Inc. New York City) have been approved for treatment of metastatic colorectal cancer but are always used in combination with standard chemotherapy regimens. In some embodiments, the cancer therapy includes one or more of the chemical therapeutic agents including thymidylate synthase inhibitors or antimetabolites such as 5-fluorouracil (5-FU), alone or in combination with other therapeutic agents.

### Driver Mutations and Driver Genes

Disclosed herein include methods, compositions, kits, and systems for predicting and/or determining clinical outcome for a combination treatment of cancer of the present disclosure, monitoring of the combination treatment, predicting and/or determining responsiveness of a subject to the combination treatment, determining the status of the cancer in a subject, and improving combination treatment outcome. The methods, compositions, kits, and systems can comprise determination of the change(s) of driver mutation(s) in driver genes.

The Cancer Genome Atlas (TCGA) Project enabled detailed characterization genetic alterations resulting in colorectal cancer by identifying a larger number of mutated genes in colorectal tumors, including well known genes, such as APC, TP53, SMAD4 and PIK3CA as well as some that are less well known, such as SOX9 or ACVR1B. A more recent study added additional putative driver genes, such as PRKCI, MAP2K4, and TGFBR2. These results highlighted the importance of several key pathways, including MAPK, WNT and TGFβ-signaling pathways. In some embodiments, the driver mutation is a mutation in one of the below 74 genes AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. In some embodiment, the driver mutations are in KRAS, APC and TP53 genes.

### KRAS

The KRAS gene (also known as Kirsten rat sarcoma viral oncogene homolog, KRAS Proto-Oncogene, GTPase, K-Ras, KRAS2) is a proto-oncogene that encodes a GTPase that is part of signal transduction pathways that regulate mitosis.

Several mutations in KRAS activate the protein and are implicated in cancer such as acute myelogenous leukemia (AML), juvenile myelomonocytic leukemia (JMML), gastric cancer, colorectal cancer, pancreatic cancer and lung cancer. Cancers with mutant KRAS often have aggressive growth. Mutations in the KRAS genes have been found codon 12, codon 13, codon 18, codon 61, codon 117, and codon 146. The most common activating mutations in the KRAS gene are found in codons 12 and 13, including but not limited to G13D, G13D, G12V, G12D, G12A, G12R, G12S and G12C. Non-limiting examples of KRAS mutations include A18D, Q61H, and K117N. As used herein, KRAS gene mutations can comprise, for example, G12A, G12C, G12D, G12R, G12S, G12V, G13C, G13D, G13S, G13R, A18D, G61H, Q61L, Q61K, Q61R, K117N, A146T, A146V, A146P, A11V, or a combination thereof.

Drugs are in development that target KRAS G12C, but no drugs are currently available that targets KRAS activated by the other mutations. Efforts to target cancers with KRAS mutations focus on inhibiting proteins that share the same signal transduction pathways as KRAS. A genome-wide RNAi screen was completed to identify what gene(s) is necessary for KRAS-mutated tumor cells to drive tumor growth. Among the genes identified was PLK1, where inhibition of PLK1 was hypothesized to be a synthetic lethal against cancers with KRAS mutants. Synthetic lethality is when a combination of deficiencies in the expression of two or more genes leads to cell death, whereas a deficiency in only one of these genes does not. When PLK1 is inhibited in a cell with a wild-type KRAS, e.g., a non-cancerous cell, the cell remains viable. However, cell death occurs when PLK1 is inhibited in a cancer cell with a KRAS mutant.

Without being bound to any particular theory, it is believed that KRAS mutants are experiencing mitotic stress and exacerbating this stress in particular ways such that interference with PLK1 leads to stress overload and tumor cell death. KRAS mutants together with PLK1 inhibition can block the anaphase promoting complex (APC/C), which is crucial for mitosis to occur. The APC/C complex is critical in mediating the metaphase to anaphase transition. The dual block of APC/C from PLK1 inhibitor treatment is believed to cause the synthetic lethality to KRAS mutant cancer cells.

Tumors having KRAS mutations and resistant to treatment can emerge during treatment. This resistance is particularly common with anti-EGFR treatment of metastatic colorectal cancer (mCRC), where up to 50% of mCRC patients undergoing standard-of-care anti-EGFR treatment (e.g., cetuximab and/or panitumumab) with wild-type KRAS develop resistant tumors with KRAS-based mutations. Secondary treatment options are needed for those patients. There is also a need for a KRAS-based assay that can enable rapid assessment of the presence of prevalence of mutant KRAS as an early predictor of response to treatment. Methods, compositions, and kits disclosed herein can be used for treating cancer, for example colorectal cancer. Colorectal cancer (CRC) is often associated with a KRAS mutation. The standard-of-care chemotherapy for colorectal cancer is currently FOLFIRI, which is a combination of leucovorin (folinic acid), 5-fluorouracil (5-FU), and irinotecan. Bevacizumab is often combined with FOLFIRI, however, that combination has only a 4% response rate against metastatic CRC (mCRC).

### APC

The adenomatous polyposis coli (APC) gene is a tumor suppressor gene located in the human chromosome region 5q21-22. As a housekeeping gene, it consists of 15 exons and encodes a 300 kDa protein composed of 2,843 amino acids, which plays a vital role in cellular proliferation, migration, DNA repair, and chromosomal segregation. It was initially identified as the pathogenic gene in familial adenomatous polyposis (FAP), an autosomal dominant hereditary disease with numerous adenomatous polyps all over the colorectum. It has also been found that the development of CRC is initiated by the aberrant outgrowth of adenomatous polyps from the colonic epithelium that ultimately evolve into aggressive carcinomas. About 85% of sporadic colorectal cancers have been reported to harbor APC truncating mutations. The growth of the polyps is associated in most cases with alterations of both alleles of the APC gene. A first mutational hit occurs roughly in the middle of the open reading frame, generating a truncated APC molecule lacking the C-terminal half. Such truncation mutations are located in the so-called mutation cluster region (MCR). The second mutational hit involves either deletion of the second allele or a mutation that leads to the synthesis of a truncated product, almost never occurring after the MCR. Thus, colon cancer cells express at least a truncated APC molecule whose length is defined by the position of the MCR and, occasionally, an additional but shorter fragment.

As a negative regulator of Wnt signaling, APC regulates gene transcription by influencing Wnt signaling. Wnts are a family of secreted cysteine-rich glycoproteins that have been implicated in the regulation of stem cell maintenance, proliferation, and differentiation during embryonic development. Canonical Wnt signaling increases the stability of cytoplasmic β-catenin by receptor-mediated inactivation of GSK-3 kinase activity and promotes β-catenin translocation into the nucleus. The canonical Wnt signaling pathway also functions as a stem cell mitogen via the stabilization of intracellular β-catenin and activation of the β-catenin/TCF/LEF transcription complex, resulting in activated expression of cell cycle regulatory genes, such as Myc, cyclin D1, EPhrinB (EPhB) and Msx1, which promote cell proliferation.

Without the negative regulation by APC, the Wnt pathway is more active and plays a role in the development of cancer. Studies comparing tumor cells with mutations in both APC alleles to correlate levels of Wnt signaling and severity of disease in both humans and mice have aided in establishing a model in which gene dosage effects generate a defined window of enhanced Wnt signaling, leading to polyp formation in the intestine. Combinations of ‘milder’ APC mutations, associated with weaker enhancement of Wnt signaling, give rise to tumors in extra-intestinal tissues. According to this model, the nature of the germline mutation in APC determines the type of somatic mutation that occurs in the second allele.

The APC protein consisting of multiple domains, which bind to various proteins, including beta-catenin, axin, C-terminal binding protein (CtBP), APC-stimulated guanine nucleotide exchange factors (Asefs), Ras GTPase-activating-like protein (IQGAP1), end binding-1 (EB1) and microtubules. Studies using mutant mice and cultured cells demonstrated that APC suppresses canonical Wnt signaling, which is essential for tumorigenesis, development and homeostasis of a variety of cell types, including epithelial and lymphoid cells. Further studies have suggested that the APC protein functions in several other fundamental cellular processes. These cellular processes include cell adhesion and migration, organization of actin and microtubule networks, spindle formation and chromosome segregation. Deregulation of these processes caused by mutations in APC is implicated in the initiation and expansion of colon cancer. The APC protein functions as a signaling hub or scaffold, in that it physically interacts with a number of proteins relevant to carcinogenesis. Loss of APC influences cell adhesion, cell migration, the cytoskeleton, and chromosome segregation.

APC mutations cause a loss of function change in colon cancer. Missense mutations yield point mutations in APC, while truncation mutations cause the loss of large portions of the APC protein, including defined regulatory domains. A significant number of APC missense mutations have been reported in tumors originating from various tissues, and have been linked to worse disease outcome in invasive urothelial carcinomas, suggesting the functional relevance of point mutated APC protein in the development of extra-intestinal tumors.

APC mutation resulting in a change of function can influence chromosome instability in at least three manners: by diminishing kinetochore-microtubule interaction, by the loss of mitotic checkpoint function and by generating polyploid cells. For example, studies have shown that APC bound to microtubules increased microtubule stability in vivo and in vitro, suggesting a role of APC in microtubule stability. Truncated APC led to chromosomal instability in mouse embryonic stem cells, interfered with microtubule plus-end attachments, and caused a dramatic increase in mitotic abnormalities. Studies have shown that cancer cells with APC mutations have a diminished capacity to correct erroneous kinetochore-microtubule attachments, which account for the wide-spread occurrence of chromosome instability in tumors. In addition, abrogation of the spindle checkpoint function was reported with APC loss of function. Knockdown of APC with siRNA indicated that loss of APC causes loss of mitotic spindle checkpoint function by reducing the association between the kinetochore and checkpoint proteins Bub1 and BubR1. Thus, loss of APC reduces apoptosis and induces polyploidy. Polyploidy is a major source for aneuploidy since it can lead to multipolar mitosis.

A large fraction of colon cancer patients have at least one APC gene product that is truncated. Thus, truncated APC proteins can play an active role in colon cancer initiation and progression. For example, truncated APC, but not full-length APC, may activate Asef and promote cell migration. Although defects in APC occur in a high fraction of colon cancer cases, there are currently no therapeutics targeting vulnerabilities resulting from these defects.

### TP53

The tumor suppressor p53 gene (TP53), located on chromosome 17p13.1, is known as “the guardian of the genome” or “the cellular gatekeeper of growth and division.” The gene contains 11 exons and transcribes a 2.8 kb mRNA, which is translated into a 53 kDa protein. The protein encoded by TP53, p53, a 393 amino acid long phosphoprotein, acts as a key regulator of cellular growth control and plays a central role in in cell cycle arrest and apoptosis following DNA damage.

TP53 is one of the most frequently mutated genes in human cancers. The most common mutations are single base substitutions that alter protein function. Some of the mutations being oncogenic confer gain-of-function properties. Germline mutations in TP53 occur in families with Li-Fraumeni syndrome, which is associated with an increased risk of developing various cancers with an early age of onset. Somatic mutations at specific residues have been associated with specific clinical phenotypes in different type of cancer. The reported frequency of mutations in TP53 in CRC is about 50%. These mutations affect mainly five “hotspot” codons, including 175, 245, 248, 273 and 282.

In addition to being frequently mutated in cancers, the gene is also highly polymorphic. The most investigated polymorphism in codon 72 of exon 4. The polymorphism of codon 72 is located in a proline-rich region of the protein, which has been known for long to be important for the growth suppression and apoptotic functions. Thus, a number of studies have investigated the role of the presumptively functional codon 72 polymorphism in modulation of cancer risk. As indicated by several meta-analyses, Arg72Pro variant has been observed to be involved in susceptibility to breast, lung and gastric cancers but not cervical cancer. Several investigations have confirmed the association between the Arg72Pro polymorphism and the modulation of CRC risk including studies on risk of adenoma. One study explored the association between colon cancer and 94 SNPs in 63 genes, concluding that the GSTTJ null genotype in combination with the TP53 Pro72 allele significantly increased the risk. Another frequently studied TP53 polymorphism is 16-bp duplication in intron 3. However, there are inconsistent observations on the association between the TP53 PIN3 polymorphism and CRC risk.

Association of somatic mutations in TP53 or abnormal protein expression with poor survival or lack of response to therapy has been reported. However, the clinical significance of TP53 status remains to be controversial. The effects of TP53, and its abnormalities, on the response of tumors to cytotoxic drugs, radiation and chemoradiation are also complex. Some research suggests that it is, perhaps, unrealistic to expect a straightforward relationship between any mutation in TP53 and the response to treatment with chemotherapy.

In some embodiments, the driver mutation is a mutation in one of the below 74 driver genes AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. In some embodiments, at least one of the one or more of the driver mutations is a mutation in the 74 driver genes. In some embodiments, one or more of the driver mutations are mutations in the 74 driver genes.

The driver mutation or at least one of the one or more driver mutations can be in a gene selected from the group consisting of AR, BRAF, CCND1, CCND2, CCNE1, COK4, COK6, EGFR, ERBB2, FGFR1, FGFR2, KIT, KRAS, MET, MYC, POGFRA, PIK3CA, and RAF1. In some embodiments, the driver mutation or at least one of the one or more driver mutations is in a gene selected from the group consisting of ALK, FGFR2, FGFR3, NTRK1, RET, and ROS1. In some embodiments, the driver mutation or at least one of the one or more driver mutations is in a gene selected from the group consisting of APC, AR, ATM, BRAF, BRCA1, BRCA2, CDK4, CDK6, CDK12, EGFR, ERBB2, HRAS, KIT, KRAS, MAPK1, MAPK3, MET, MYC, NRAS, PIK3CA, PTEN, RB1, STK11, and TP53.

There have been studies on the association between germline and somatic variants in colorectal cancer. However, the germline and somatic variants are not strongly correlated in predicting risks of CRC, except only a variant identified with some degree of association. For example, a variant (rs78963230) located within a CNA region associated with TLR3 also associated with a somatic non-silent mutation within gene FBXW7. Moreover, the somatic variant rs2302274 located in CDX1/PDGFRB frequently gained/lost in colorectal tumors was associated with overall CRC risk.

The driver mutation can be a somatic mutation in driver genes. In some embodiments, the driver genes are KRAS, APC, and TP53. The somatic mutation can be a substitution, an insertion, a deletion, an amplification, a fusion or a combination thereof. In some embodiments, the somatic mutation is a point mutation (e.g., single-nucleotide variant (SNV)). In some embodiments, the somatic mutation is an insertion-deletion mutation (indel).

### PLK Inhibitors, Dosing and Pharmacokinetics

Polo-like kinases (PLK) are a family of five highly conserved genes from the serine/threonine protein kinase superfamily. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies. PLK1 inhibition induces G2-M-phase arrest with subsequent apoptosis in cancer cells, and has emerged as a promising targeted therapy. Non-limiting examples of PLK1 inhibitors include onvansertib, BI2536, volasertib (BI 6727), GSK461364, HMN-176, HMN-214, AZD1775, CYC140, rigosertib (ON-01910), MHLN0905, TKM-080301, TAK-960, Ro3280, and any combination thereof.

Onvansertib (also known as PCM-075, NMS-1286937, NMS-937, “compound of formula (I)” described in U.S. Pat. No. 8,927,530; IUPAC name 1-(2-hydroxyethyl)-8-{[5-(4-methylpiperazin-1-yl)-2-(trifluoromethoxy) phenyl]amino}-4,5-dihydro-1H-pyrazolo[4,3-h]quinazoline-3-carboxamide) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. For example, it shows high potency in proliferation assays having low nanomolar activity on a large number of cell lines, both from solid as well as hematologic tumors. Onvansertib is the first PLK1 specific ATP competitive inhibitor administered by oral route to enter clinical trials with proven antitumor activity in different preclinical models.

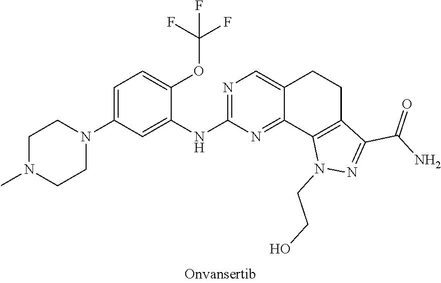 [SMILES: CN1CCN(c2ccc(OC(F)(F)F)c(Nc3ncc4c(n3)-c3c(c(C(N)=O)nn3CCO)CC4)c2)CC1]

Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone, and shows prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over previous PLK inhibitors, including high selectivity for PLK1 only, oral availability and half-life of about 24 hours.

A Phase 1 dose-escalation study with onvansertib has been conducted in adult subjects with advanced/metastatic solid tumors at a single study site in the U.S. The primary objective of that study was to determine a maximum tolerated dose (MTD) of onvansertib in adult subjects with advanced/metastatic solid tumors. Secondary objectives of the study were to define antitumor activity. In that study, a recommended phase 2 dose of 24 mg/m² was established and 5 of 16 evaluable patients had stable disease.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in PCT Application No. PCT/US2021/013287 titled “Circulating Tumor DNA as a Biomarker for Leukemia Treatment” and filed on Jan. 13, 2021, the content of which is incorporated herein by reference in its entirety.

The cancer treatment of the present disclosure can comprise administration of a PLK1 inhibitor (e.g., onvansertib) to a subject with cancer for a desired duration in a cycle, two cycles, or more cycles. The desired duration in each cycle can independently be one, two, three, four, five, six, seven, eight, nine, ten, or more days. The cycle can be, for example, at least 20 days, 21 days, 22 days, 23 days, 24 days, 25 days, or more, in length. For example, a single cycle of the treatment can comprise administration of the PLK1 inhibitor (e.g., onvansertib) for four days, five days, six days, seven days, eight days, nine days, ten days, eleven days, twelve days, thirteen days, fourteen days, fifteen days, sixteen days, seventeen days, eighteen days, nineteen days, twenty days, or more in a cycle (e.g., a cycle of at least 21 days (e.g., 21 to 28 days)). In some embodiments, the treatment can comprise administration of the PLK1 inhibitor (e.g., onvansertib) for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days. In some embodiments, the duration of administration of the PLK1 inhibitor in one cycle can be different from the duration of the administration of the PLK1 inhibitor in one or more other cycles. For example, the PLK1 inhibitor can be administered to the subject for 10 days (e.g., day 1 to day 5 in the first 14 days and day 1 to day 5 in the last 14 days in a 28-day cycle) for the first cycle, and for 14 days in the second cycle (e.g., day 1 to day 7 in the first 14 days and day 1 to day 7 in the last 14 days in a 28-day cycle). The length of each of the cycles can vary. For example, cycle 1 can be 28 days, and cycle 2 can be 21 days.

The cancer treatment disclosed herein can comprise administration of the PLK1 inhibitor (e.g., onvansertib) at, or at about, 12 mg/m²-90 mg/m², for example, as a daily dose. For example, the treatment can comprise daily administration of the PLK1 inhibitor (e.g., onvansertib) at, or at about, 8 mg/m², 10 mg/m², 12 mg/m², 14 mg/m², 15 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the daily dose of the PLK1 inhibitor (e.g., onvansertib) is 12 mg/m², 15 mg/m², 18 mg/m², or 24 mg/m². In some embodiments, the daily dose of the PLK1 inhibitor (e.g., onvansertib) is 15 mg/m². The daily dose of the PLK1 inhibitor (e.g., onvansertib) for each cycle of treatment can vary. For example, the daily dose of the PLK1 inhibitor (e.g., onvansertib) for the first cycle can be 12 mg/m², and the daily dose of the PLK1 inhibitor (e.g., onvansertib) for the second cycle can be increased to, for example, 15 mg/m². In some embodiments, the daily dose of the PLK1 inhibitor (e.g., onvansertib) for the second cycle can then be increased to, for example, 18 mg/m². Without being bound by any particular theory, it is believed that the mg/m² doses disclosed herein are Body Surface Area (BSA) based doses corresponding to flat doses in the range of 20 to 45 mg.

A maximum concentration (Cₘₐₓ) of the PLK1 inhibitor (e.g., onvansertib) in a blood of the subject (during the treatment or after the treatment) when the PLK1 inhibitor is administered alone or in combination with one or more additional cancer therapeutics (e.g., FOLFIRI and bevacizumab) can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with one or more additional cancer therapeutics (e.g., FOLFIRI and bevacizumab) can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with one or more additional cancer therapeutics (e.g., FOLFIRI and bevacizumab) can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with one or more additional cancer therapeutics (e.g., FOLFIRI and bevacizumab) can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with one or more additional cancer therapeutics (e.g., FOLFIRI and bevacizumab) can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the one or more additional cancer therapeutics (e.g., FOLFIRI and bevacizumab) can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with one or more additional cancer therapeutics (e.g., FOLFIRI and bevacizumab) can be from about 10 hours to about 60 hours. For example, the elimination half-life (T_{1/2}) of the PLK1 inhibitor (e.g., onvansertib) in a blood of the subject when the PLK1 inhibitor is administered alone or in combination with one or more additional cancer therapeutics (e.g., FOLFIRI and bevacizumab) can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

### Additional Cancer Therapeutics or Therapy

Methods, compositions and kits disclosed herein can be used for treating cancer, for example colorectal cancer. A method for treating colorectal cancer can comprise administrating a PLK1 inhibitor (e.g., onvansertib) and bevacizumab to a subject (e.g., a patient) in need thereof. The method can comprise administering a therapeutically effective amount of the PLK1 inhibitor and a therapeutically effective amount of bevacizumab. The treatment can comprise administration of at least one additional cancer therapeutics or cancer therapy. The treatment can comprise administration a therapeutically effective amount of at least one additional cancer therapeutics or cancer therapy. The PLK1 inhibitor and the at least one additional cancer therapeutics or cancer therapy can, for example, co-administered simultaneously or sequentially. Bevacizumab and the at least one additional cancer therapeutics or cancer therapy can, for example, co-administered simultaneously or sequentially.

Non-limiting examples of the additional cancer therapeutics or cancer therapy can include surgery, chemotherapy, radiation therapy (including external-beam, stereotactic, and intraoperative radiation therapy and brachytherapy), bone marrow transplant, immunotherapy, targeted drug therapy, cryoablation, or radiofrequency ablation. Where the cancer is colorectal cancer, examples of treatments include surgery, radiofrequency ablation, cryoablation, radiation therapy, chemotherapy (including medications comprising capecitabine, 5-fluorouracil (5-FU), irinotecan, oxaliplatin, trifluridine/tipiracil), targeted therapy (including anti-angiogenesis therapy using, for example bevacizumab, regorafenib, ziv-aflibercept, or ramucirumab; immunotherapy using, for example pembrolizumab, nivolumab, or ipilimumab; and PLK1 inhibitors).

The additional cancer therapeutics or cancer therapy can be a chemotherapy. For example, the additional cancer therapeutics or cancer therapy can comprise FOLFIRI, FOLFOX, XELOX (CAPOX), or FOLFOXIRI. Chemotherapy regimens using fluorouracil are standard treatment for advanced colorectal cancer. Fluorouracil is a pyrimidine analog and antimetabolite, which incorporates into the DNA molecule and stops synthesis, thereby, preventing replication of cancer cells. Examples of these regimens include FOLFOX and FOLFIRI. The additional cancer therapeutics or cancer therapy can comprise a therapeutically effective amount of FOLFIRI. FOLFIRI is a chemotherapeutical cocktail, containing leucovorin (folinic acid), fluorouracil and irinotecan hydrochloride. Leucovorin is a vitamin B derivative and increases the cytotoxicity of fluorouracil in this combination. Irinotecan is a topoisomerase inhibitor, which prevents DNA from uncoiling and duplicating. FOLFIRI is often used in combination with other therapeutical reagents (e.g., bevacizumab) to improve efficacy and response rate.

FOLFOX is a chemotherapy regimen for treatment of colorectal cancer, made up of folinic acid, fluorouracil and oxaliplatin (Eloxatin). FOLFOX can be broken down into other subtypes such as FOLFOX-4, FOLFOX-6, and FOLFOX-7 depending on how these three drugs are administered. FOLFOX is usually used to treat colorectal cancer. It can also be used to treat pancreatic cancer and certain other cancers. FOLFOX is typically used as an adjuvant treatment (in addition to the primary therapy) for advanced cancers. However, FOLFOX can also be used as a first-line therapy for colorectal adenocarcinoma, which is the most common type of colon cancer. In this combination, oxaliplatin shows synergy with fluorouracil, with little toxicity overlap.

For decades, fluorouracil had been the only drug with demonstrated activity against colorectal cancer, commonly used in combination with leucovorin. Oxaliplatin and capecitabine are two relatively novel drugs used in the treatment of colorectal cancer. These drugs have been found to act synergistically, both in vivo and in vitro. The chemotherapy combination of oxaliplatin and capecitabine is known as XELOX, which is highly active in metastatic colorectal cancer (mCRC). Capecitabine has demonstrated high efficacy as first-line treatment for MCRC. It is an oral fluoropyrimidine that was rationally designed to generate FU preferentially at the tumor site, via a three-step enzymatic process that exploits the significantly higher activity of thymidine phosphorylase (TP) in tumors, compared with healthy tissue. Capecitabine causes less adverse effects, such as diarrhea, stomatitis, nausea, alopecia, and neutropenia, leading to less neutropenic fever/sepsis and associated hospitalizations compared to the combination therapy of leucovorin and fluorouracil (e.g., FOLFIRI, and FOLFOX), although hand-foot syndrome (HFS) occurred more frequently with capecitabine administration. Oxaliplatin is a third-generation cisplatin analog, and an organoplatinum complex, and is usually classified as alkylating agent although it is not capable of actually adding alkyl groups to DNA. Oxaliplatin is an integral component of the various fluorouracil regimens (e.g., FOLFOX), which have become a standard treatment for metastatic and node-positive colorectal cancer.

Another combination therapy of oxaliplatin and fluorouracil is known as FOLFOXIRI, containing leucovorin, fluorouracil, oxaliplatin and irinotecan. FOLFOXIRI is also an approved chemotherapy regimen for the treatment of advanced colorectal cancer and is often given with bevacizumab.

The additional cancer therapeutics can comprise FOLFIRI, bevacizumab, abiraterone, FOLFOX, an anti-EGFR agent, a KRAS directed inhibitor, gemcitabine, abraxane, nanoliposomal irinotecan, 5-FU, or a combination thereof. In some embodiments, the PLK inhibitor and the cancer therapeutics or cancer therapy are co-administered simultaneously or sequentially.

### Methods for Predicting/Determining Responsiveness

Mutations in the driver genes can be detected in a biological sample (including but not limited to a bodily fluid (e.g., a blood sample)) from a subject of interest (e.g., a subject with colorectal cancer, a subject is in partial remission of colorectal cancer, or a subject suspected to have colorectal cancer). For example, the mutations can be detected in the circulating tumor cells (CTCs), circulating tumor DNA (ctDNA), PBMC, or a combination thereof, obtained from plasma fraction, serum fraction, or both, of a blood sample. In some embodiments, the bodily sample is whole blood, serum, plasma, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine, or any combination thereof. In some embodiment, the CTCs and/or ctDNA is obtained from blood and fractions thereof. A sample can be in the form originally isolated from a subject or can have been subjected to further processing to remove or add components, such as cells, or enrich for one component relative to another. Thus, it can be advantageous in some embodiments to analyze plasma or serum containing ctDNA. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may not have cancer or a detectable cancer symptom. The subject may have been treated with one or more cancer therapy, e.g., any one or more of chemotherapies, antibodies, vaccines or biologics. The subject may be in remission. The subject may be suspected to have cancer or any cancer-associated genetic mutations/disorders.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., ctDNA. Others are released from healthy cells. ctDNA can be obtained from a bodily fluid without the need to perform an in vitro cell lysis step, and thus presents a non-invasive option for genomic analysis. Provided herein include methods, compositions, kits and systems for detecting and/or analyzing cell free nucleic acids (e.g., ctDNA) in bodily fluid (e.g., peripheral blood) for clinical outcome prediction/determination. The methods can comprise combined analysis of single cells and cell-free nucleic acids. Provided herein include methods utilizing ctDNA from whole blood (e.g., plasma and/or serum) for therapeutic monitoring, and minimal/molecular residual disease determination.

Various assays (e.g., sequencing assays) can be used to detect and analyze ctDNA or nucleic acids from CTCs. The methods provided herein can comprise isolation and analysis of ctDNA from the blood (e.g., plasma and/or serum) of a subject of interest (e.g., a subject with colorectal cancer), employing the use of molecular barcoding and sequencing as a readout. The method can comprise isolating plasma and ctDNA from intact cell-depleted blood. The method can comprise centrifugation to generate plasma and extraction of nucleic acids from plasma, followed by library prep with barcoding, sequencing, and then analysis. The ctDNA, for example, can be obtained from a plasma sample by known methods, and can be analyzed by methods including but not limited to polymerase chain reaction (PCR) and next generation sequencing (NGS). In some embodiments, the ctDNA is analyzed using high-throughput NGS.

The ctDNA can carry one or more types of mutations, for example, germline mutations, somatic mutations, or both. Germline mutations refer to mutations existing in germline DNA of a subject. Somatic mutations refer to mutations originating in somatic cells of a subject, e.g., cancer cells. In some embodiments, the ctDNA carries somatic mutations. In some embodiments, the mutations are not germline mutations. In some embodiments, the mutations are not clonal haematopoiesis of indeterminate potential (CHIP) mutation. In some embodiments, the mutation can be a colorectal cancer-associated mutation.

The ctDNA from a subject can carry one or more mutations in one or more genes, including AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. For example, the ctDNA from the subject can carry mutations in KRAS, APC, and TP53. In some embodiments, at least one of the one or more of the driver mutations is a mutation in the above 74 driver genes. In some embodiments, one or more of the driver mutations are mutations in the above 74 driver genes.

The driver mutation or at least one of the one or more driver mutations can be in a gene selected from the group consisting of AR, BRAF, CCND1, CCND2, CCNE1, COK4, COK6, EGFR, ERBB2, FGFR1, FGFR2, KIT, KRAS, MET, MYC, POGFRA, PIK3CA, and RAF1. In some embodiments, the driver mutation or at least one of the one or more driver mutations is in a gene selected from the group consisting of ALK, FGFR2, FGFR3, NTRK1, RET, and ROS1. In some embodiments, the driver mutation or at least one of the one or more driver mutations is in a gene selected from the group consisting of APC, AR, ATM, BRAF, BRCA1, BRCA2, CDK4, CDK6, CDK12, EGFR, ERBB2, HRAS, KIT, KRAS, MAPK1, MAPK3, MET, MYC, NRAS, PIK3CA, PTEN, RB1, STK11, and TP53.

Exemplary amounts of ctDNA in a biological sample (e.g., plasma or serum) before amplification range from about 1 fg to about 1 g, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of ctDNA molecules. The method can comprise obtaining 1 femtogram (fg) to 200 ng ctDNA. The ctDNA can have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

ctDNA can be isolated from bodily fluids (e.g., plasma) through a fractionation or partitioning step in which ctDNA, as found in solution, are separated from intact cells and other non-soluble components of the bodily fluid. Partitioning can include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. After such processing, samples can include various forms of nucleic acid including double stranded DNA and single stranded DNA. In some embodiments, single stranded DNA can be converted to double stranded forms so that they are included in subsequent processing and analysis steps.

There are provided, in some embodiments, methods, reagents, compositions, and systems for analyzing complex genomic material while reducing or eliminating loss of molecular characteristic (e.g., epigenetic or other types of structural) information that is initially present in the complex genomic material. In some embodiments, molecular tags can be used to track ctDNA and determine genetic modifications (e.g., SNVs, indels, gene fusions and copy number variations). The method for detecting and analyzing ctDNA can comprise: classifying one or more variant properties derived from the sequence reads generated from one or more sequencing assays on the isolated ctDNA as a true cancer-associated variant, a CHIP-associated variant, and/or a mutation of unknown origin. The method can comprise: adjusting the prediction score, the MRD score, and/or the efficacy score based on the classification of the one or more variant properties derived from the sequence reads generated from one or more sequencing assays on the isolated ctDNA.

Methods, compositions, kits and systems disclosed herein can be applied to different types of subjects. For example, the subject can be a subject receiving a cancer treatment, a subject at cancer remission (e.g., partial remission), a subject has received one or more cancer treatment, or a subject suspected of having cancer. The subject can have a stage I cancer, a stage II cancer, a stage III cancer, and/or a stage IV cancer. The cancer can comprise a solid cancer, for example colorectal cancer, including metastatic colorectal cancer (mCRC). The cancer can comprise one or more mutations in genes, including AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. For example, the cancer comprises mutations in KRAS, APC, and TP53. The methods disclosed herein can comprise: administering a therapeutic intervention to the subject. The therapeutic intervention can comprise a different therapeutic intervention, an antibody, an adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, or any combination thereof. The therapeutic intervention can be administered at a time when the subject has an early-stage cancer, and wherein the therapeutic intervention is more effective that if the therapeutic intervention were to be administered to the subject at a later time.

As disclosed herein, useful information such as the responsiveness of the subject to a treatment, effectiveness and/or clinical benefits of the cancer treatment can be obtained by evaluating ctDNA from more than one plasma sample, for example plasma collected (a) before or at treatment, and (b) at least once after treatment has started. In those embodiments, the second or subsequent samples can be taken at any time after treatment has started, e.g., after the first round of treatment, after multiple rounds of treatment, or after the colorectal cancer is no longer detected in order to determine whether the colorectal cancer has returned. In some embodiments, the blood sample (e.g., plasma) collected after treatment is collected after a first round of treatment, e.g., at least 10, 15, 20, 21, 28, or 35 days, or any number of days in between or outside of those numbers, after the start of treatment.

Analyzing ctDNA can comprise analyzing ctDNA for one or more markers (e.g., ctDNAs comprising variant/mutant alleles). For example, ctDNA can be analyzed to assess variant allele frequency (VAF), change in the mean VAF, total mutation burden, and/or development of new mutations in driver genes (e.g., KRAS, APC, and TP53) in a subject with cancer (e.g., colorectal cancer). The subject can be a subject to be selected for a cancer treatment, a subject that is undergoing a cancer treatment, or a subject that has undergone a cancer treatment. In some embodiments, the ctDNA analysis measures the amount of a mutation in one or more driver genes (e.g., KRAS, APC, and TP53) in the ctDNA. In some embodiments, the ctDNA analysis measures the amount of a mutation in at least three driver genes, including KRAS, APC, and TP53, in the ctDNA. In some embodiments, the ctDNA analysis measures mutation allelic frequencies (MAF) of diver genes in the ctDNA. For the methods disclosed herein, analyzing ctDNA from a subject can comprise detecting variant allele frequency (VAF) in the ctDNA, and a change in mean VAF of two or more driver genes at different time points can indicate the subject as responsive to the cancer treatment. For example, analyzing ctDNA from a subject can comprise detecting a change (e.g., decrease) in mean VAF of KRAS, APC, and TP53 genes prior to and at the end of a cycle of a cancer treatment, which can indicate the subject as responsive to the cancer treatment.

As described herein, a decrease in MAF during treatment, e.g., when comparing the MAF before treatment with the MAF after the first cycle of treatment, is indicative or predictive of clinical response. For example, a decrease in mean ctDNA MAF of at least three driver genes, including KRAS, APC, and TP53, can be indicative of a positive clinical outcome. The decrease in mean MAF of driver genes (e.g., KRAS, APC, and TP53) can be 25%, 30%, 35%, 40%, 45%, 50%, 55%, 60%, 65%, 70%, 75%, 80%, 90%, or a number or a range between any two of these values. The decrease in MAF of driver genes can be at least, or at least about, 25%, 30%, 35%, 40%, 45%, 50%, 55%, 60%, 65%, 70%, 75%, 80%, or 90%. MAF determination can be used in the methods, compositions, kits and systems described herein for ctDNA analysis herein to determine effectiveness of treatment and future treatment of cancer (e.g., colorectal cancer). In some embodiments, these methods can be used to decide whether to continue a treatment, e.g., when there is a decrease in MAF during the treatment, or change the treatment, e.g., if there is not a decrease in MAF during treatment. In some embodiments, clinically meaningful reduction in MAF is 25%-40%, which is indicative of clinical response.

Methods for Determining Efficacy and/or Improving Outcome for Cancer Treatment

Disclosed herein include methods, compositions, kits, and systems for predicting/determining clinical outcome for a cancer treatment, monitoring of the cancer treatment, predicting/determining responsiveness of a subject to the cancer treatment, determining the cancer status of a subject, and improving cancer treatment outcome. The treatment comprises administering, a PLK1 inhibitor (e.g., onvansertib) and bevacizumab to the subject. For example, the treatment can be a combination treatment using a PLK1 inhibitor (e.g., onvansertib), bevacizumab and one or more additional cancer therapeutics (e.g., FOLFIRI).

The methods, compositions, kits and systems can be used to guide the cancer treatment, provide treatment recommendations, reduce or avoid unnecessary ineffective treatment for patients. For example, ctDNA and/or CTCs can be analyzed to predict/determine clinical outcome for cancer treatment comprising administration of a PLK1 inhibitor and bevacizumab of the present disclosure, monitor the combination treatment, predict/determine responsiveness of a subject to the treatment, determine cancer status in a subject, improve cancer treatment outcome, guide cancer treatment, provide cancer treatment recommendations, and/or to reduce or avoid ineffective cancer treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT Application No. PCT/US2021/013287, the content of which is incorporated herein by reference in its entirety.

The first time point for determining driver mutation(s) (e.g., determining variant allele frequency of at least three driver genes) can be, for example, prior or immediately prior, to the cancer treatment (that is, pre-dosing). The at least one of the one or more additional time points can be, for example, at the end, or right before the end, of or after at least a cycle (e.g. the first cycle, the second cycle, the third cycle, or any of the subsequent cycles) of the cancer treatment. In some embodiments, the cycle of the cancer treatment is the first cycle of the cancer treatment. In some embodiments, the first time point is prior or immediately prior to a first cycle of the cancer treatment. In some embodiments, the one or more additional time points are at the end, or right before the end, of a second cycle, a third cycle, a fourth cycle, and/or a fifth cycle of the treatment. In some embodiments, the one or more additional time points are after a second cycle, a third cycle, a fourth cycle, and/or a fifth cycle of the cancer treatment. In some embodiments, the first cycle of the cancer treatment is immediately prior (e.g., one day, two days, three days, four days, or five days) to the second cycle of the cancer treatment. In some embodiments, the method comprises continuing the cancer treatment to the subject if the subject is indicated as responsive to the cancer treatment. In some embodiments, the method comprises discontinuing the cancer treatment to the subject and/or starting a different cancer treatment to the subject if the subject is not indicated as responsive to the cancer treatment.

In some embodiments, the first time point is prior or immediately prior to the onset of the cancer treatment (e.g., a combination treatment), and at least one of the one or more additional time points are at the end of or after at least a cycle of the treatment. In some embodiments, the cycle of the combination treatment is the first cycle of the treatment. In some embodiments, the first time point is prior or immediately prior to a first cycle of the treatment, and the one or more additional time points are at the end of or after a second cycle of the treatment. In some embodiments, the first cycle of the combination treatment is immediately prior to the second cycle of the treatment. In some embodiments, the method comprises continuing the treatment to the subject if the subject is indicated as responsive to the treatment. In some embodiments, the method comprises discontinuing the combination treatment to the subject and/or starting a different treatment to the subject if the subject is not indicated as responsive to the treatment.

The first sample can comprise ctDNA and/or CTCs from the subject at various time points, for example before the treatment or during the treatment. In some embodiments, the first sample comprises ctDNA and/or CTCs from the subject before the treatment (for example, immediately before the first cycle of the treatment). In some embodiments, the first sample comprises ctDNA and/or CTCs from the subject before the second cycle of the treatment (for example, after the completion of the first cycle of the treatment and immediately before the second cycle of the treatment). The additional samples can comprise ctDNA and/or CTCs from the subject during and/or after the treatment. In some embodiments, the additional samples comprise ctDNA from the subject right before the end of and/or after the treatment. In some embodiments, the additional samples comprise ctDNA and/or CTCs from the subject right before the end of and/or after the first cycle, the second cycle, the third cycle, the fourth cycle, and/or the fifth cycle of the treatment.

Some embodiments comprise detecting mean variant allele frequency of somatic mutation(s) of at least three driver genes, including KRAS, APC, and TP53, in the ctDNA. In some embodiments, analyzing the ctDNA comprises detecting mean variant allele frequency of driver genes in the ctDNA obtained from the subject at a first time point in a first sample, detecting mean variant allele frequency of driver genes in the ctDNA obtained from the subject at one or more additional time points in one or more additional samples, and determining the difference of the mean variant allele frequency in ctDNA between the first and at least one of the one or more additional samples, an increase in the mean variant allele frequency of the driver genes at the additional sample(s) relative to the first sample indicates that the subject is not a responder for the cancer treatment. In some embodiments, the method comprises discontinuing the cancer treatment and/or starting an additional cancer treatment to the subject if the subject is indicated as a non-responder to the cancer treatment. The additional treatment can be the same or different from the current or prior treatment.

The variant allele frequency of driver mutation(s) in ctDNA can be determined, for example, by mean variant allele frequency of driver mutation(s) of at least three genes (e.g., KRAS, APC, and TP53) in the ctDNA in each of the first sample and one or more additional samples (e.g., plasma samples). The ctDNA can be analyzed using, for example, PCR, next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include a method of determining responsiveness of a subject to a cancer treatment, comprising treating a subject with cancer and the treating comprises administering a PLK1 inhibitor and bevacizumab to the subject; detecting change(s) in the mean level of driver mutation(s) in the subject, and determining the responsiveness of the subject to the cancer treatment based on the change(s) detected in mean level of driver mutation(s). Change(s) in mean level of driver mutation(s) in the subject can be determined by detecting one or more mutations in at least three driver genes (e.g., KRAS, APC, and TP53) in the subject (1) during the subject is treated for cancer, (2) before the subject is treated for cancer, (3) after the subject is treated for cancer, or a combination thereof. Examples of driver genes include AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. For example, somatic mutation(s) of KRAS, APC, and TP53 genes can be detected in the subject immediate before the subject is administered with the PLK1 inhibitor (i.e., pre-dosing) and bevacizumab, and detected again (once or multiple times) during the first cycle of the cancer treatment. In some embodiments, the driver mutations are detected at the end of the first cycle of the cancer treatment.

In some embodiments, detecting change(s) in mean level of mutation(s) of driver genes in the subject comprises detecting mean level of mutations of driver genes two or more times in the subject. For example, the mean level of mutations of driver genes can be detected twice, three times, four times, five times, and each time independently at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28, or a number or a range between any two of these values, of the cancer treatment. In some embodiments, the detection of mean level of mutations of driver genes occurs at day 5, 7, 14 or 28 of the cancer treatment or the first cycle of the cancer treatment. In some embodiments, the mean level of mutations of driver genes is detected every day during the cancer treatment.

Change(s) in mean level of mutations of driver genes can be, or comprise, change(s) in mean level of mutations of driver genes during the subject is treated for cancer, change(s) in mean level of mutations of driver genes from before the subject is treated for cancer to during the subject is treated for cancer, or a combination thereof. For example, the mean level of mutations of driver genes in the subject can change after the subject starts to receive the cancer treatment (as compared to pre-dosing). In some embodiments, the subject develops different mean level of mutations of driver genes or different mean variant allele frequency of driver genes during the cancer treatment. The variant allele frequency can be, for example, mutant allelic frequency (MAF). The variant allele frequency of driver genes can be determined by total mutation count, mean variant allele frequency, number of KRAS mutation alleles per unit (e.g., ml) of sample (e.g., plasma sample), or both. In some embodiments, the variant allele frequency of driver genes is determined by mean variant allele frequency. In some embodiments, the subject has one or more mutations in the driver genes before being treated with the PLK1 inhibitor. In some embodiments, the subject does not have mutations in the driver genes before being treated with the PLK1 inhibitor and bevacizumab.

In some embodiments, detecting change(s) in mean level of driver mutation(s) of at least three driver genes (e.g., KRAS, APC, and TP53) in the subject comprises detecting change(s) in mean level of driver mutation(s) of at least three driver genes (e.g., KRAS, APC, and TP53) in a biological sample from the subject, or derivative thereof. The biological sample can be, or comprise, a bodily fluid, whole blood, plasma, one or more tissues, one or more cells, or a combination thereof. The bodily fluid can be, or comprise, blood, plasma, urine, or a combination thereof. The biological sample can comprise ctDNA, CTC, or a combination thereof.

In the methods described herein, determining the responsiveness of the subject comprises determining if the subject is a responder of the treatment, if the subject is or is going to be in complete recover (CR), or if the subject is or is going to be in partial remission (PR). In some embodiments, determining the responsiveness of the subject comprises determining if the subject has a partial response to the treatment, if the subject has a complete response to the treatment, if the subject has a stable disease (SD) status, or if the subject has a progressive disease (PD) status.

The cancer treatment with the PLK1 inhibitor can be maintained, for example, if the change in mean MAF of at least three driver genes is a decrease of at least 25%, at least 50%, or at least 75%. Such a decrease can, for example, be detected at the end of cycle 1 of the cancer treatment or at day 1 of cycle 2 of the cancer treatment. In some embodiments, the cancer treatment is maintained if there is at least a 50% decrease in mean MAF of at least three driver genes. In some embodiments, the cancer treatment is maintained if there is at least a 75% decrease in mean MAF of at least three driver genes. In some embodiments, the cancer treatment with the PLK1 inhibitor and bevacizumab is maintained if the mean level of driver mutations (e.g., somatic mutations) of at least three driver genes (e.g., KRAS, APC, and TP53) in the samples decreases to below 0.01% or below 0.001% of the driver genes (e.g., KRAS, APC, and TP53) in the sample.

The cancer treatment with the PLK1 inhibitor and bevacizumab can be modified or discontinued if the change in mean MAF of at least three driver genes is a decrease of less than 50%, less than 25%, or less than 10%. Such a decrease can, for example, be detected at the end of cycle 1 of the cancer treatment or at day 1 of cycle 2 of the cancer treatment. In some embodiments, the cancer treatment is modified or discontinued if there is less than 50% decrease in mean MAF of at least three driver genes. In some embodiments, the cancer treatment is modified or discontinued if there is less than 25% decrease in mean MAF of at least three driver genes. In some embodiments, the cancer treatment with the PLK1 inhibitor and bevacizumab is modified or discontinued if the mean level of driver mutations of at least one driver genes in the samples does not decrease to below 0.01% or below 0.001% of the at least one driver genes in the sample. In some embodiments, detecting change(s) in the mean level of driver mutation(s) of at least three driver genes in the subject comprising detecting one or more driver mutations of at least three driver genes emerged in the subject after the subject being treated with the PLK1 inhibitor and bevacizumab.

Also disclosed herein includes a method of improving outcome of a cancer treatment. The method comprises: detecting mean variant allele frequency of at least three driver genes (e.g., KRAS, APC, and TP53) in a subject at a first time point in a first sample, wherein the first time point is before the subject starts the cancer treatment, or during the cancer treatment, and wherein the cancer treatment comprises administering a PLK1 inhibitor and bevacizumab to the subject; detecting mean variant allele frequency of at least three driver genes (e.g., KRAS, APC, and TP53) in the subject at one or more additional time points in one or more additional samples after the subject, wherein the at least one of the one or more additional time points is during the cancer treatment; determining the difference of the mean variant allele frequency of at least three driver genes (e.g., KRAS, APC, and TP53) between the first and the one or more additional samples, wherein a decrease in the variant allele frequency in at least one of the one or more additional samples relative to the first sample indicates the subject as responsive to the cancer treatment; and continuing the cancer treatment to the subject if the subject is indicated as responsive to the cancer treatment, or discontinuing the cancer treatment to the subject and/or starting a different cancer treatment to the subject if the subject is not indicated as responsive to the cancer treatment. Examples of driver genes include AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. The first time point can be before the subject starts the cancer treatment. In some embodiments, at least two of the additional time points are during the cancer treatment.

Also disclosed herein includes a method of treating cancer, comprising: treating a subject with cancer, wherein the treating comprises administering a PLK1 inhibitor and bevacizumab to the subject; determining a decrease, relative to a mean variant allele frequency of at least three driver genes (e.g., KRAS, APC, and TP53) in a first sample of the subject obtained at a first time point before the subject receives the cancer treatment or during the cancer treatment, in a mean variant allele frequency of at least three driver genes (e.g., KRAS, APC, and TP53) in a second sample of the subject obtained at a second time point after the subject starts receiving the cancer treatment; and continuing with the cancer treatment. In some embodiments, the first time point is prior or immediately prior to the cancer treatment. In some embodiments, the first time point is during the cancer treatment. For example, the first time point is at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28 of the cancer treatment. In some embodiments, the first time point is at day 5, 7, 14, or 28 of the cancer treatment.

In some embodiments, at least one of the one or more additional time points is during the cancer treatment. For example, the first time point is at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28 of the cancer treatment. In some embodiments, at least one of the one or more additional time points is at day 5, 7, 14, or 28 of the cancer treatment. In some embodiments, the one or more additional time points is during the cancer treatment. For example, the first time point is at day 1, day 2, day 3, day 5, day 7, day 10, day 11, day 12, day 13, day 14, day 15, day 16, day 17, day 18, day 19, day 20, day 21, day 22, day 23, day 24, day 25, day 26, day 27, day 28 of the cancer treatment. In some embodiments, the one or more additional time points is at day 5, 7, 14, or 28 of the cancer treatment. In some embodiments, the first time point and at least one of the one or more additional time points are during the first cycle of the cancer treatment. In some embodiments, at least one of the one or more additional time points are during the first cycle of the cancer treatment, and at least one of the one or more additional time points are during the second cycle of the cancer treatment.

As disclosed herein, the mean variant allele frequency is, in some embodiments, mutant allelic frequency (MAF). In some embodiments, detecting mean variant allele frequency in at least three genes (e.g., KRAS, APC, and TP53) comprises detecting mean variant allele frequency in at least three genes (e.g., KRAS, APC, and TP53) in a biological sample from the subject, or derivative thereof. The biological sample can be, or comprise, a bodily fluid, whole blood, plasma, one or more tissues, one or more cells, or a combination thereof. In some embodiments, the bodily fluid comprises blood, plasma, urine, or a combination thereof. In some embodiments, the biological sample comprises circulating tumor DNA (ctDNA), circulating tumor cell (CTC), or a combination thereof. In some embodiments, droplet digital PCR (ddPCR), polymerase chain reaction (PCR) or next generation sequencing (NGS) is used to determine mean MAF of at least three genes (e.g., KRAS, APC, and TP53).

The subject can have one or more mutations in the driver genes before being treated with the PLK1 inhibitor and bevacizumab. In some embodiments, the subject does not have mutations in the driver genes before being treated with the PLK1 inhibitor and bevacizumab. In some embodiments, the subject has received one or more prior cancer treatment. The cancer can be advanced, metastatic, refractory, or relapsed. The cancer can be colorectal cancer (e.g., metastatic colorectal cancer), pancreatic cancer, leukemia, lung cancer, or a combination thereof.

The driver genes can include AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. The driver mutation can be a somatic mutation. The somatic mutation can be a substitution, an insertion, a deletion, an amplification, a fusion or a combination thereof. In some embodiments, the somatic mutation is a point mutation (e.g., single-nucleotide variant (SNV)). In some embodiments, the somatic mutation is an insertion-deletion mutation (indel).

In some embodiments, the PLK1 inhibitor is onvansertib. In some embodiments, the treatment comprises administration of onvansertib for ten days in a cycle of 28 days. For example, the treatment can comprise administration of onvansertib for five days in the first 14 days and five days in the second 14 days in a cycle of 28 days. In some embodiments, the cancer treatment comprises administering to the subject at least one additional cancer therapeutics or cancer therapy, including but not limited to FOLFIRI, FOLFOX, XELOX (CAPOX), FOLFOXIRI, or a combination thereof. In some embodiments, the PLK inhibitor and the additional cancer therapeutics or cancer therapy are co-administered simultaneously or sequentially. In some embodiments, the PLK inhibitor and bevacizumab are co-administered simultaneously or sequentially. In some embodiments, bevacizumab and the additional cancer therapeutics or cancer therapy are co-administered simultaneously or sequentially.

In some embodiments, the cancer treatment comprises one or more cycles, and change(s) in mean level of driver mutation(s) of at least three driver genes or mean variant allele frequency of at least three driver genes is detected before, during and/or after each cycle of the leukemia treatment. Each cycle of treatment can be at least 21 days, for example, from about 21 days to about 28 days.

As disclosed herein, periodic measurement of mean level of driver mutations of at least three driver genes in ctDNA, for example in a bodily fluid, of a cancer patient can provide early indication of the effectiveness of treatment being administered to the patient. Examples provided herein showed that where PLK1 inhibition by onvansertib is an effective treatment, in combination with standard-of-care FORFIRI and bevacizumab, for metastatic colorectal cancer.

The combination treatment can cause a reduction of mean level of mutation of at least three driver genes in plasma cell-free DNA to below the level of detection (0.001%) within a month (in some cases within a week) of the start of the treatment. This reduction can correlate with radiographic response in those patients. Thus, periodic measuring of mean level of driver mutations of at least three driver genes in ctDNA of cancer patients is useful for quickly determining treatment effectiveness.

Provided herein include a method comprising: (a) treating a patient with colorectal cancer, and (b) periodically sampling a bodily fluid from the patient and measuring the mean level of mutation of at least three driver genes in cell-free DNA in the bodily fluid.

These methods are useful for evaluating a treatment for any cancer. Nonlimiting examples include leukemia, lung cancer, colorectal cancer, and pancreatic cancer. In some embodiments, the cancer is colorectal cancer. In some of those embodiments, the cancer is metastatic colorectal cancer.

Any treatment of a cancer can be evaluated using these methods. Non-limiting examples include surgery, chemotherapy, radiation therapy (including external-beam, stereotactic, and intraoperative radiation therapy and brachytherapy), bone marrow transplant, immunotherapy, targeted drug therapy, cryoablation, or radiofrequency ablation. Where the cancer is colorectal cancer, examples of treatments include surgery, radiofrequency ablation, cryoablation, radiation therapy, chemotherapy (including medications comprising capecitabine, 5-FU, irinotecan, oxaliplatin, trifluridine/tipiracil), targeted therapy (including anti-angiogenesis therapy using, for example bevacizumab, regorafenib, ziv-aflibercept, or ramucirumab; immunotherapy using, for example pembrolizumab, nivolumab, or ipilimumab; and PLK1 inhibitors).

The treatment can comprise administration of a PLK1 inhibitor. Nonlimiting examples include onvansertib, BI2536, volasertib (BI 6727), GSK461364, HMN-176, HMN-214, AZD1775, CYC140, rigosertib (ON-01910), MLN0905, TKM-080301, TAK-960 or Ro3280. In various embodiments, the PLK1 inhibitor is onvansertib.

Any bodily fluid that would be expected to have nucleic acids can be utilized in these methods. Non-limiting examples of bodily fluids include peripheral blood, serum, plasma, urine, lymph fluid, amniotic fluid, and cerebrospinal fluid. In various embodiments the bodily fluid is blood, plasma or urine.

The method can be applied to a patient having any cancer having driver mutations in driver genes now known or later discovered. Driver genes can be AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. The driver mutation can be a somatic mutation. In some embodiments, the driver genes are KRAS, APC, and TP53. The somatic mutation can be a substitution, an insertion, a deletion, an amplification, a fusion or a combination thereof. In some embodiments, the somatic mutation is a point mutation (e.g., single-nucleotide variant (SNV)). In some embodiments, the somatic mutation is an insertion-deletion mutation (indel).

In some examples, after treatment of mCRC with onvansertib, FOLFIRI and bevacizumab, the driver mutants can become nondetectable (less than 0.001% of KRAS) within one week of the start of treatment. In these methods, the samples can be taken at any time in relation to the beginning of the treatment. In some embodiments, a sample is taken prior to, or at, the beginning of the treatment. In some embodiments, a sample is taken more than once after the start of treatment, e.g., at least twice within one after administration of the treatment. In some embodiments, a sample is taken within one week after the beginning of treatment. In some embodiments, at least two samples of the bodily fluid are taken within one month of starting the treatment. In further embodiments, a sample is taken within one month from the beginning the treatment.

The driver mutations in the sample can be measured by any method now known or later discovered. Nonlimiting examples include any PCR and any next-generation sequencing (NGS) method. In some embodiments, the method is high-throughput NGS. With those methods, any parameter of the driver mutations in the sample can be measured, for example mean MAF of somatic mutations of at least three driver genes (as in Example).

The methods, composition and kits disclosed herein can be utilized to make treatment decisions, e.g., whether to maintain the treatment or modify the treatment. The particular level of driver mutations in the sample that directs the treatment recommendation can be determined for any particular treatment and cancer without undue experimentation, optionally taking into consideration any other particular factors that could affect the recommendation, e.g., possible interactions with other medications taken by the patient, other patient disorders that could affect the effectiveness or tolerance of the treatment, etc.

In some embodiments of these methods, (i) if the mean level of driver mutations of at least three driver genes in the samples decreases to below a set percentage of driver genes in the sample, the treatment is maintained, or (ii) if the mean level of driver mutations of at least three driver genes in the samples does not decrease to below the set percentage of driver genes in the sample, the treatment is modified. In these embodiments, the percentage of the driver genes in the sample that is the threshold between maintaining and modifying the treatment can be determined by consideration of any number of factors, for example the sensitivity of the assay and past results with other patients. In some embodiments, the percentage decrease, above which treatment modification is indicated, is 0.1%, 0.05%, 0.01%, 0.005%, 0.001%, or any percentage in between or outside those percentages; for example 0.01% or 0.001%.

When the method indicates that the treatment should be modified, the modified treatment can comprise any of the treatments discussed above. In some embodiments of these methods, the driver mutations of the driver genes emerged in a resistant tumor of the cancer after the patient was treated for the cancer having wild-type driver genes. In some of these embodiments, the cancer is mCRC.

### Kits

Disclosed herein include kits for determining responsiveness of a subject to a cancer treatment, kits for improving outcome of a cancer treatment, and kits for treating cancer. The kit, in some embodiments, comprises: a PLK1 inhibitor (e.g., onvansertib) or a pharmaceutically acceptable salt, solvate, stereoisomer thereof, bevacizumab and a manual providing instructions for performing one or more of the steps for one or more methods disclosed herein.

In some embodiments, the kit comprises: a PLK1 inhibitor (e.g., onvansertib) or a pharmaceutically acceptable salt, solvate, stereoisomer thereof, bevacizumab, and manual providing instructions for performing one or more steps of the method disclosed herein for determining responsiveness of a subject to a cancer treatment. For example, the method can comprise: treating a subject with cancer, wherein the treating comprises administering a PLK1 inhibitor and bevacizumab to the subject; detecting change(s) in the mean level of somatic mutation(s) of at least three driver genes (e.g., KRAS, APC, and TP53) in the subject, and determining the responsiveness of the subject to the cancer treatment based on the change(s) detected in the mean level of somatic mutation(s) of at least three driver genes (e.g., KRAS, APC, and TP53). In some embodiments, detecting change(s) in mean level of somatic mutation(s) of at least three driver genes (e.g., KRAS, APC, and TP53) in the subject comprises detecting mean level of somatic mutation(s) of at least three driver genes (e.g., KRAS, APC, and TP53) in the subject (1) during the subject is treated for cancer, (2) before the subject is treated for cancer, (3) after the subject is treated for cancer, or a combination thereof. Detecting change(s) in mean level of somatic mutation(s) of at least three driver genes (e.g., KRAS, APC, and TP53) can comprise detecting mean variant allele frequency of at least three driver genes (e.g., KRAS, APC, and TP53), for example mean MAF of KRAS, APC, and TP53 genes. The cancer treatment with the PLK1 inhibitor and bevacizumab can be maintained if the change in mean MAF of the at least three driver genes (e.g., KRAS, APC, and TP53) is a decrease of at least 25%, at least 50%, or at least 75%. In some embodiments, the decrease is detected at the end of cycle 1 of the cancer treatment or at day 1 of cycle 2 of the cancer treatment. The cancer treatment with the PLK1 inhibitor and bevacizumab can be, for example, modified or discontinued if the change in mean MAF of the at least three driver genes (e.g., KRAS, APC, and TP53) is a decrease of less than 50%, less than 25%, or less than 10%. In some embodiments, the decrease is detected at the end of cycle 1 of the cancer treatment or at day 1 of cycle 2 of the cancer treatment.

The kit can comprise: a PLK1 inhibitor (e.g., onvansertib) or a pharmaceutically acceptable salt, solvate, stereoisomer thereof, bevacizumab, and manual providing instructions for performing one or more steps of the method disclosed herein for improving outcome of a cancer treatment. For example, the method can comprise: detecting mean variant allele frequency of least three driver genes (e.g., KRAS, APC, and TP53) in a subject at a first time point in a first sample, wherein the first time point is before the subject starts the cancer treatment, or during the cancer treatment, and wherein the cancer treatment comprises administering a PLK1 inhibitor and bevacizumab to the subject; detecting mean variant allele frequency of least three driver genes (e.g., KRAS, APC, and TP53) in the subject at one or more additional time points in one or more additional samples after the subject, wherein the at least one of the one or more additional time points is during the cancer treatment; determining the difference of the mean variant allele frequency of least three driver genes (e.g., KRAS, APC, and TP53) between the first and the one or more additional samples, wherein a decrease in the mean variant allele frequency in at least one of the one or more additional samples relative to the first sample indicates the subject as responsive to the cancer treatment; and continuing the cancer treatment to the subject if the subject is indicated as responsive to the cancer treatment, or discontinuing the cancer treatment to the subject and/or starting a different cancer treatment to the subject if the subject is not indicated as responsive to the cancer treatment. The first time point can be, for example, before the subject starts the cancer treatment. In some embodiments, at least two of the additional time points are during the cancer treatment. In some embodiments, the kit comprises: a PLK1 inhibitor (e.g., onvansertib) or a pharmaceutically acceptable salt, solvate, stereoisomer thereof, bevacizumab, and manual providing instructions for performing one or more steps of the method disclosed herein for treating cancer. For example, the method can comprise: treating a subject with cancer, wherein the treating comprises administering a PLK1 inhibitor and bevacizumab to the subject; determining a decrease, relative to a mean variant allele frequency of least three driver genes (e.g., KRAS, APC, and TP53) in a first sample of the subject obtained at a first time point before the subject receives the cancer treatment or during the cancer treatment, in a mean variant allele frequency of least three driver genes (e.g., KRAS, APC, and TP53) in a second sample of the subject obtained at a second time point after the subject starts receiving the cancer treatment; and continuing with the cancer treatment. Detecting mean variant allele frequency of least three driver genes (e.g., KRAS, APC, and TP53) can be, for example, detecting mean MAF of KRAS, APC, and TP53 genes.

Driver genes can be AKT1, ALK, APC, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, KRAS, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TP53, TSC1, and VHL. The driver mutation can be a somatic mutation. In some embodiments, the driver genes comprise or are KRAS, APC, and TP53. The somatic mutation can be a substitution, an insertion, a deletion, an amplification, a fusion or a combination thereof. In some embodiments, the somatic mutation is a point mutation (e.g., single-nucleotide variant (SNV)). In some embodiments, the somatic mutation is an insertion-deletion mutation (indel).

The instructions can comprise instructions for administering the PLK1 inhibitor at 9 mg/m²-90 mg/m², for example at from 9 mg/m² to 24 mg/m². For example, the instructions can be for administering the PLK1 inhibitor at 12 mg/m², 15 mg/m², or 18 mg/m².

Some embodiments provided herein provide a method comprising (a) treating a patient for a cancer (e.g., colorectal cancer), and (b) periodically sampling a bodily fluid from the patient and measuring the mean level of mutations of at least three driver genes (e.g., KRAS, APC, and TP53) in cell-free DNA in the bodily fluid. The cancer can be leukemia, lung cancer, colorectal cancer (e.g., metastatic colorectal cancer), or pancreatic cancer. In some embodiments, the treatment comprises administration of a polo-like kinase 1 (PLK1) inhibitor, including but are not limited to one or more of onvansertib, BI2536, volasertib (BI 6727), GSK461364, HMN-176, HMN-214, AZD1775, CYC140, rigosertib (ON-01910), MLN0905, TKM-080301, TAK-960, and Ro3280. In some embodiments, the PLK1 inhibitor is onvansertib. The bodily fluid can be blood, plasma or urine. The driver mutations of the at least three driver genes can be measured, for example, in at least two samples of the bodily fluid that are taken within one month of starting the treatment.

The treatment can be modified, in some embodiments, (i) if the mean level of mutations of at least three driver genes (e.g., KRAS, APC, and TP53) in the samples decreases to below 0.01% of the driver genes in the sample, the treatment is maintained, or (ii) if the mean level of mutations of at least three driver genes (e.g., KRAS, APC, and TP53) in the samples does not decrease to below 0.01% of the driver genes in the sample, or both (i) and (ii). The treatment can be maintained, in some embodiments, (i) if the mean level of mutations of at least three driver genes (e.g., KRAS, APC, and TP53) in the samples decreases to below 0.001% of the driver genes in the sample, the treatment is maintained, or (ii) if the mean level of mutations of at least three driver genes (e.g., KRAS, APC, and TP53) in the samples does not decrease to below 0.001% of the driver genes in sample, or both (i) and (ii). In some embodiments, the decrease in the mean level of mutations of at least three driver genes (e.g., KRAS, APC, and TP53) is determined on samples taken within one month of starting the treatment. In some embodiments, the driver mutation(s) emerged in a resistant tumor of the cancer after the patient was treated for the cancer having wild-type KRAS. The cancer can be metastatic colorectal cancer.

Also provide includes a method comprising (a) treating a patient for colorectal cancer (e.g., metastatic colorectal cancer), and (b) periodically sampling a bodily fluid from the patient and measuring the mean level of mutations of at least three driver genes (e.g., KRAS, APC, and TP53) in cell-free DNA in the bodily fluid, wherein the treatment comprises administration of a PLK1 inhibitor, for example onvansertib, and bevacizumab. In some embodiments, the driver mutations of at least three driver genes (e.g., KRAS, APC, and TP53) emerged in a resistant tumor of the cancer after the patient was treated for the cancer.

### EXAMPLES

Some aspects of the embodiments discussed above are disclosed in further detail in the following examples, which are not in any way intended to limit the scope of the present disclosure.

### Example 1

Early Decreases in Allele Frequency (MAF) of Driver Genes Predict Clinical Benefit of the PLK1 Inhibitor Onvansertib in Combination with FOLFIRI/Bevacizumab in Second-Line Treatment of Metastatic Colorectal Carcinoma (mCRC)

Second-line (2L) treatments, such as chemotherapy in combination with targeted agents, have a poor prognosis. For example, the overall response rate (ORR) can be as low as 5%-13%, the progression-free survival (PFS) about 5.7 months, and overall survival (OS) about 11.5 months.

KRAS is mutated in about 50% of colorectal cancer (CRC) patients. To-date, RAS-directed therapies have been unsuccessful. The majority of KRAS mutations are considered to be undruggable and the covalent inhibitors targeting KRAS G12C mutation have shown limited activity in CRC, while KRAS G12C mutation represents only 8% of KRAS mutations in CRC. Therefore, efforts have been devoted to the development of effective second-line treatment in KRAS-mutated mCRC.

Alternative strategies to inhibit KRAS include targeting synthetic lethal partners of mutant KRAS (i.e., proteins that are essential in KRAS-mutant but not wild-type cells). Onvansertib has been shown to be a promising therapeutic option for KRAS-mutated mCRC. Onvansertib is an oral and highly-selective PLK1 inhibitor. PLK1, a key regulator of the cell cycle, is overexpressed in CRC cells and associated with adverse clinical features. A genome-wide RNAi screen identified PLK1 inhibition to be synthetic lethal to CRC cells with mutant KRAS.

Onvansertib demonstrated potent anti-tumor activity as single agent and showed synergy in combination with irinotecan and with 5-FU in the HCT-116 KRAS-mutant CRC xenograft model. A phase 1b/2 clinical trial (NCT03829410) has also been conducted to test the anti-tumor activities of onvansertib in human patients. Patients meeting the following key eligibility criteria were included in the current study: (1) having mCRC with KRAS mutation determined in a CLIA-certified lab; and (2) failed or experienced intolerance to first-line treatment of fluoropyrimidine and oxaliplatin with or without bevacizumab. The patients were given a Treatment in a 28-day cycle. During this treatment cycle, FOLFIRI/bevacizumab were given on Days 1 and 15, and onvansertib was given on Days 1-5 and 15-19. The efficacy of this treatment was determined by overall response rate (ORR) following RECIST v1.1 standard, Progression-free survival (PFS), and changes in circulating tumor DNA (ctDNA).

Correlative studies were also carried out, aiming at assessing the association between early changes in ctDNA and clinical response (e.g., ORR and PFS) to the combination therapy of onvansertib and FOLFIRI/bevacizumab.

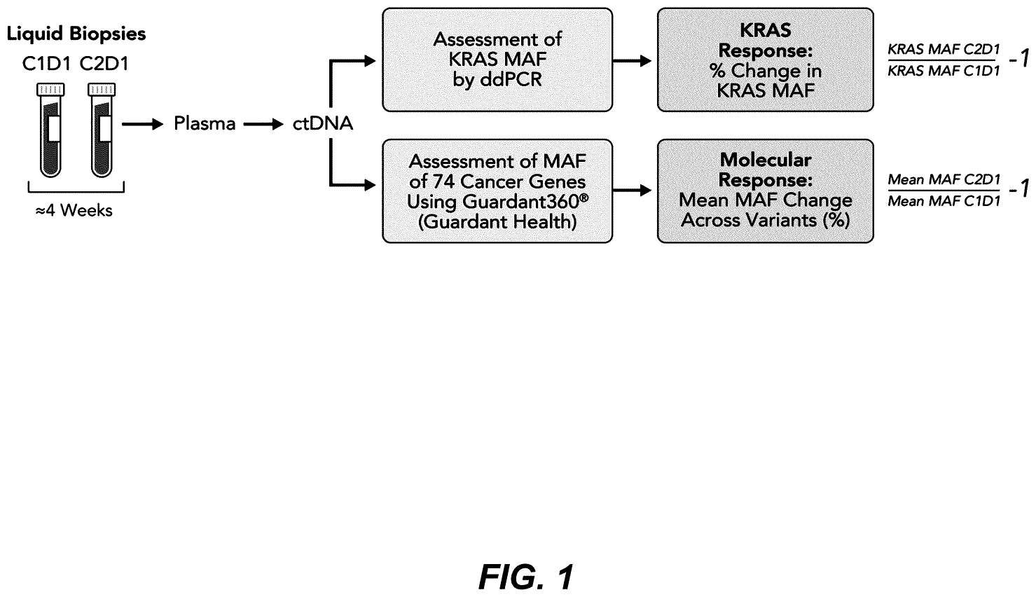

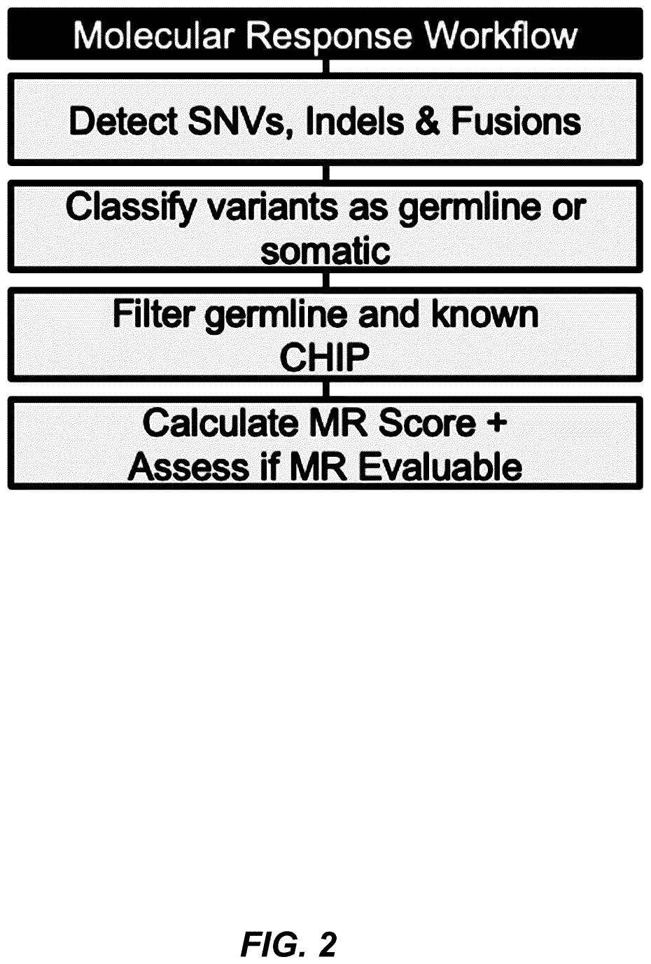

In this study, blood samples were collected at baseline (C1D1) and after 1 cycle of treatment (C2D1, about 4 weeks) to measure changes in ctDNA (FIG. 1). KRAS mutant allele frequency (MAF) was assessed by digital droplet PCR (ddPCR). Guardant360® assay, an NGS-based liquid biopsy panel covering 74 cancer genes, was used to measure the mean MAF of somatic SNVs, insertions/deletions, and gene fusions. The workflow of assessing molecular response using the panel of 74 cancer genes is illustrated in FIG. 2.

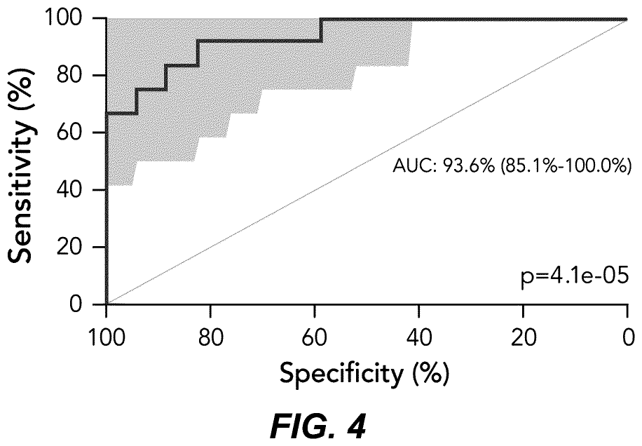

Changes in KRAS MAF were assessed in 29 patients, based on which best threshold for clinical response prediction was determined. A receiver-operating characteristic (ROC) analysis was performed to determine the best threshold in % KRAS MAF change to predict clinical response (CR/PR) (FIG. 4). % KRAS MAF change was calculated as the ratio of KRAS MAF on day one of the second cycle to KRAS MAF on day one of the first cycle. Of the 29 patients, 12 had a PR, 15 SD and 2 PD as best response. Thus, the ROC analysis included 12 responders (12 patients with PR) and 17 non-responders (15 patients with SD and 2 patients with PD).

The best threshold in % KRAS MAF change to predict clinical response was determined to be −90%. Decrease of at least 90% in KRAS MAF after the 1^{st} cycle of treatment predicted clinical response with 91.7% sensitivity and 82.4% specificity.

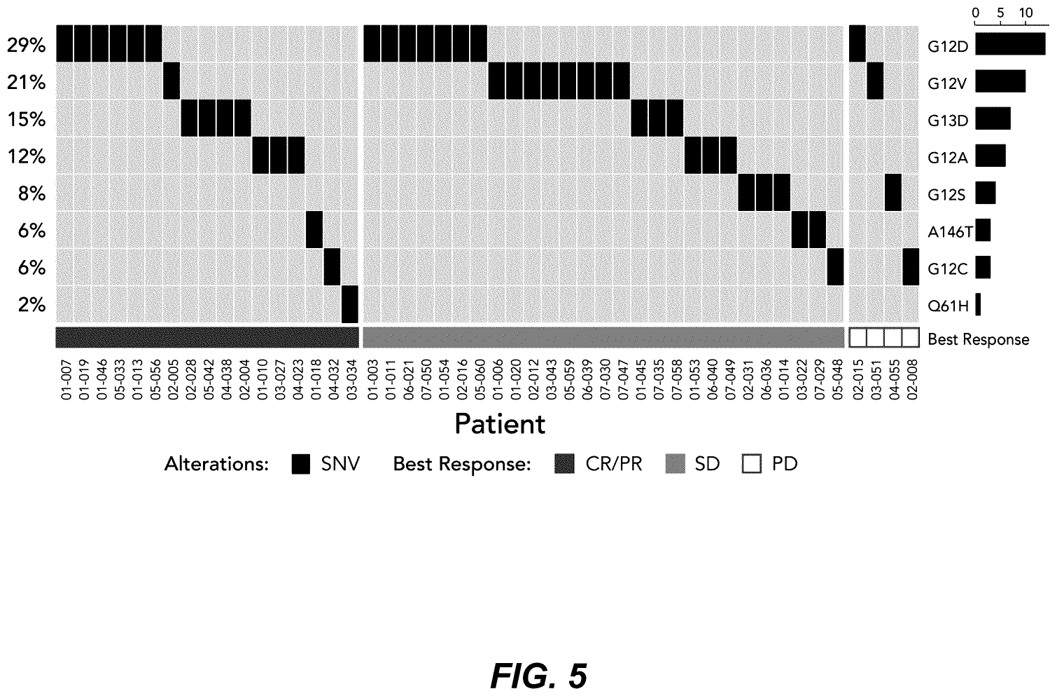

Efficacy of the combination therapy (onvansertib and FOLFIRI/bevacizumab) and association of KRAS mutations with clinical responses were also determined. Clinical responses (including CR/PR) were observed across different KRAS variants (FIG. 5). The efficacy of the combination therapy is summarized in Table 2 below. In Table 2, patients evaluable for efficacy (n) refers to the number of patients who received at least 1 cycle of treatment; disease control rate is the percentage of patients with CR, PR and SD; median duration of response [CI] is defined as time between first response and progression; NR means not reached; and CI means 95% confidence intervals.

TABLE 2
EFFICACY OF ONVANSERTIB AND FOLFIRI/BEVACIZUMAB
IN ALL PHASE 1B/2 PATIENTS

| | Patients | | | | Median |
|---|---|---|---|---|---|
| | Evaluable | | Disease | | Duration of |
| Patients | for | | Control | Median | Response |
| Treated | Efficacy | ORR | Rate | PFS [CI] | [CI] |
| (n) | (n) | (%) | (%) | (months) | (months) |
| 50 | 48 | 35.4 | 91.7 | 9.3 [7.6-13.5] | 11.7 [8.9-NR] |

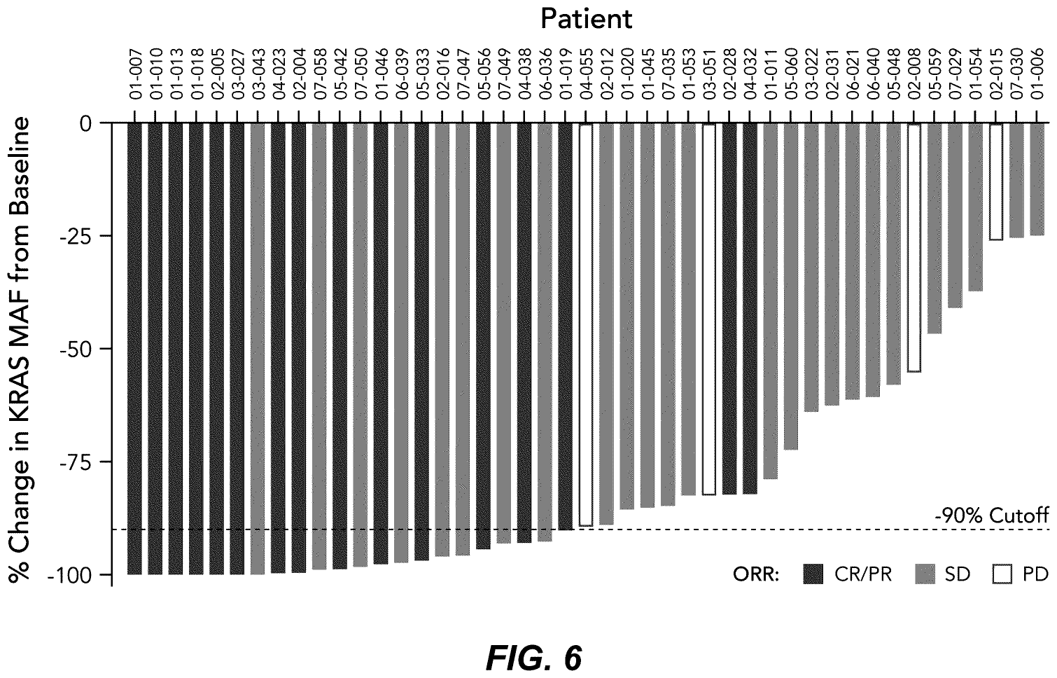

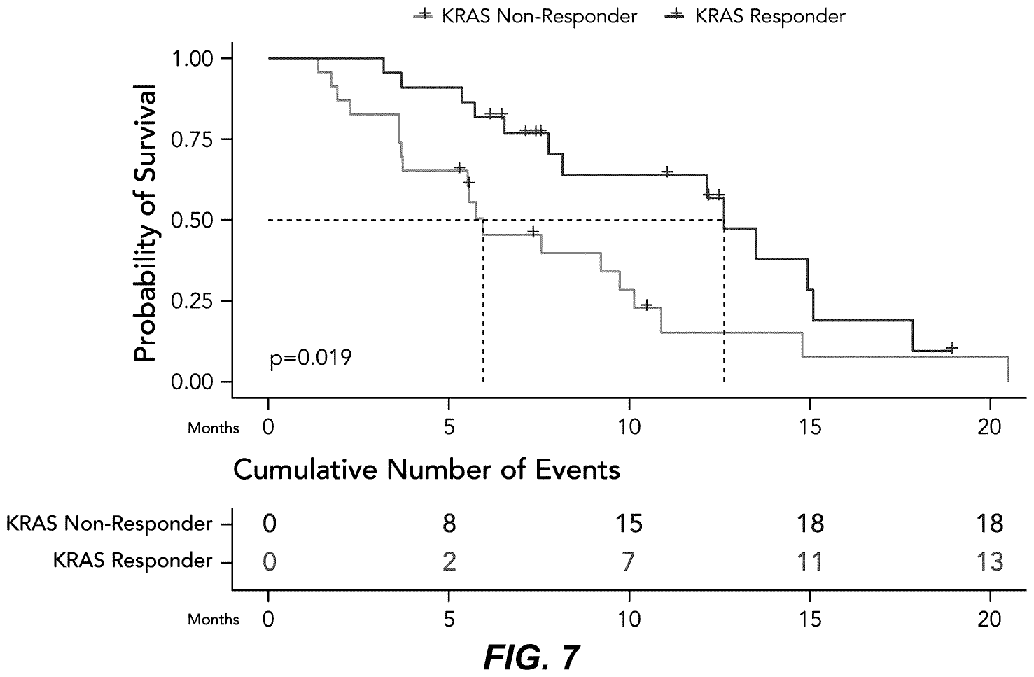

In determining whether early changes in KRAS MAF predicts clinical response, 45 patients were evaluable for KRAS response (including the 29 patients of the ROC analysis described above). Patients with C1D1 and C2D1 plasma samples and detectable baseline KRAS MAF were defined as evaluable. Based on the ROC analysis, KRAS responders were defined as patients with a ≥90% decrease in KRAS MAF after 1 cycle of treatment, as assessed by ddPCR. In this evaluation, 22 (49%) patients were determined to be KRAS responders. KRAS responders showed significantly higher ORR and longer PFS than KRAS non-responders (Table 3 and FIG. 6-FIG. 7). For example, KRAS responders have ORR of 63.6% and PFS of 12.6 months, while KRAS non-responders have ORR of 8.7% (odd ratio (OR)=16.2 [CI 3.5-131.4], p=0.00014) and PFS of 6.0 months (p=0.019). In Table 3, NR means not reached.

TABLE 3
EFFICACY OF ONVANSERTIB AND FOLFIRI/BEVACIZUMAB
IN KRAS RESPONDERS AND NON-RESPONDERS

| | KRAS | | KRAS | |
|---|---|---|---|---|
| | Responders | | Non-Responders | |
| Patients (n) | 22 | (49%) | 23 | (51%) |
| CR + PR (n) | 14 | | 2 | |
| ORR (%) | 63.6 | | 8.7 | |
| Median PFS [CI] (months) | 12.6 | [8.2-NR] | 6.0 | [3.7-10.9] |

Clinical response was also predicted by changes in MAF using Guardant360® assay. In this evaluation, 29 patients of the Phase 1b/2, with available C1D1 and C2D1 plasma samples, were evaluated for ctDNA changes using Guardant360®. Molecular Responders were defined as patients with a decrease of at least 90% in mean MAF (the 90% threshold) after 1 cycle of treatment. Using the 90% threshold, 16 (55%) patients were determined to be Molecular Responders. Molecular Responders also showed significantly higher ORR and longer PFS than Molecular Non-responders (Table 4). For example, Molecular Responders have ORR of 56.3%, while Molecular Non-responders have ORR of 7.7% (OR=12.7 [CI 1.8-366.0], p=0.0082). With respect to PFS, Molecular Responders have 12.2 months, while Molecular Non-responders have 5.6 months (p=0.0015). In Table 4, NR means not reached.

TABLE 4
EFFICACY OF ONVANSERTIB AND FOLFIRI/BEVACIZUMAB
IN MOLECULAR RESPONDERS AND NON-RESPONDERS

| | |
|---|---|
| Molecular | Molecular |
| Responder | Non-Responder |

| Patients (n) | 16 | (55%) | 13 | (45%) |
|---|---|---|---|---|
| CR + PR (n) | 9 | | 1 | |
| ORR (%) | 56.3 | | 7.7 | |
| Median PFS [CI] (months) | 12.2 | [8.2-NR] | 5.6 | [3.6-NR] |

Although both KRAS response and molecular response can be used to identify a subset of patients with increased clinical benefit, molecular response can be a more accurate test. A comparison of the performance of KRAS response and molecular response is summarized in Table 5 below.

TABLE 5
PERFORMANCE OF KRAS RESPONSE
AND MOLECULAR RESPONSE

| | Patient | | | | |
|---|---|---|---|---|---|
| | 45 | 28 subset | | | |
| Test | KRAS response | | | Molecular response | |
| Threshold | −90% | −92.85% | −90% | −92.85% | −90% |
| Sensitivity | 87.5% | 77.8% | 77.8% | 80.0% | 90.0% |
| Specificity | 72.4% | 73.7% | 68.4% | 94.7% | 73.7% |
| PPV | 63.6% | 58.3% | 53.8% | 88.9% | 64.3% |
| NPV | 91.3% | 87.5% | 86.7% | 90.0% | 93.3% |
| Accuracy | 77.8% | 75.0% | 71.4% | 89.7% | 79.3% |

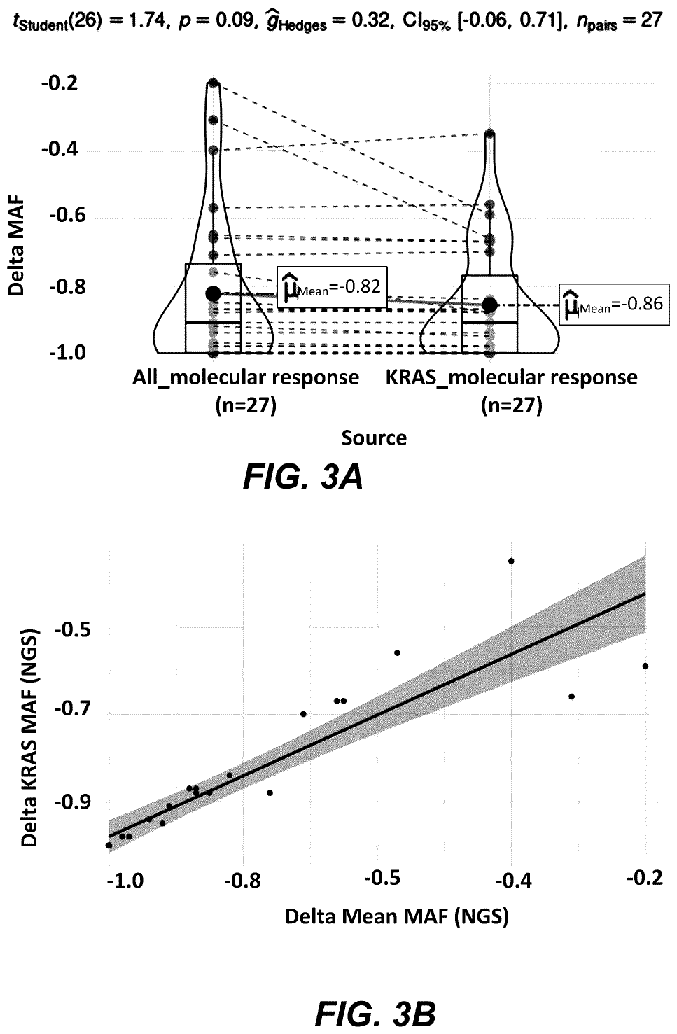

Within the context of NGS, a comparison of Delta MAF of Molecular Response with KRAS-only response shows no significant difference (FIG. 3A and FIG. 3B). However, there are a few cases where KRAS response does not represent the overall molecular response of the patient, again highlighting the benefit of using a multi-target analysis (e.g., NGS) (FIG. 3A and FIG. 3B).

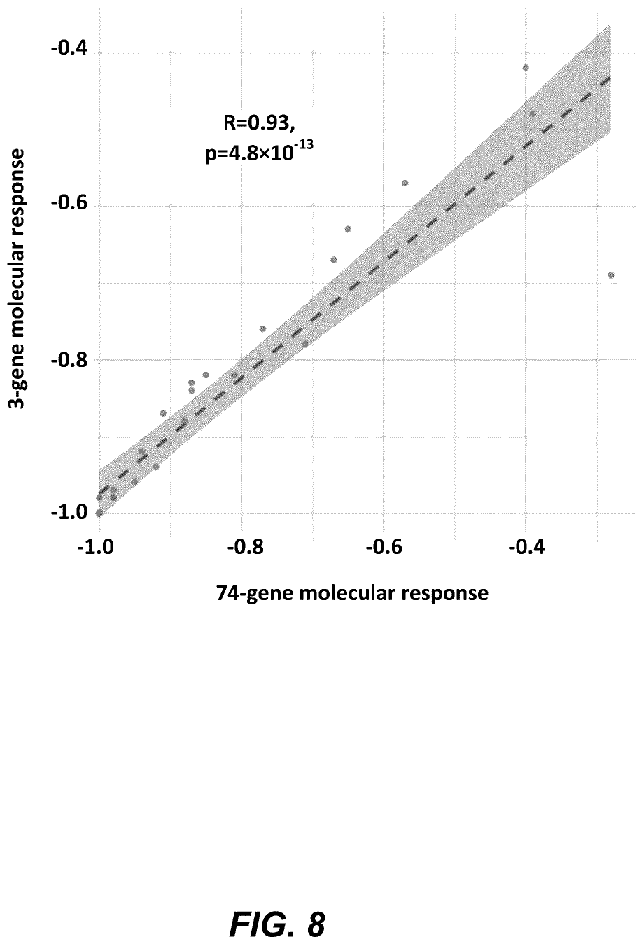

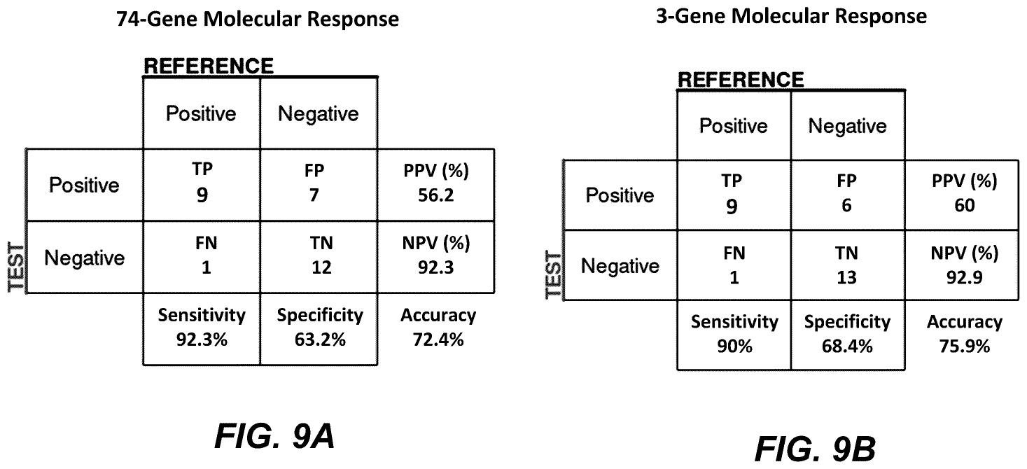

A comparison of the performance of the 74-gene molecular response test with a targeted 3-gene MR molecular response test was also carried out. The molecular response, and its ability to predict clinical benefit is similar when comparing the 74-gene panel to the use of the 3 most prevalent genes (KRAS, TP53 and APC) (FIG. 8-FIG. 9B). In FIG. 8, Pearson's correlation p=4.8×10⁻¹³.

In conclusion, onvansertib in combination with FOLFIRI/bevacizumab showed promising efficacy compared to historical control of FOLFIRI/bevacizumab second-line treatment for KRAS mutant mCRC, with an ORR of 35% and PFS of 9.3 months. Duration of response (DoR) was 11.7 months, supporting no immediate acquired resistance. The efficacy of the combination was observed across KRAS variants. Stratification of patients based on MAF changes in ctDNA after 1 treatment cycle identified a subset of patients (about 50%) with increased clinical benefits (significantly higher ORR and longer PFS). Specifically, patients with a decrease in MAF of at least 90% in a single target (KRAS determined using ddPCR) or multi-genes (74-gene panel determined using NGS) had significantly higher ORR and longer PFS. Thus, MAF changes in liquid biopsies can be used as a response biomarker to the combined therapy of onvansertib and in future clinical studies.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of predicting or determining responsiveness of a subject to a treatment for colorectal cancer, comprising:
treating the subject with colorectal cancer, wherein the treatment for colorectal cancer comprises administering a PLK1 inhibitor and bevacizumab to the subject;
detecting change(s) in mean level of somatic mutations of at least three genes in the subject, wherein the at least three genes comprise KRAS, APC and TP53 genes; and
determining the responsiveness of the subject to the treatment for colorectal cancer based on the detected change(s) in mean level of somatic mutations.

2. The method of claim 1, wherein the treatment for colorectal cancer further comprises administering a chemotherapy to the subject.

3. The method of claim 1, wherein the at least three genes further comprise one or more genes selected from the group consisting of AKT1, ALK, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TSC1, and VHL.

4. The method of any one of claims 1-3, wherein the at least three genes further comprise one or more genes selected from the group consisting of AR, BRAF, CCND1, CCND2, CCNE1, COK4, COK6, EGFR, ERBB2, FGFR1, FGFR2, KIT, MET, MYC, POGFRA, PIK3CA, and RAF1.

5. The method of any one of claims 1-4, wherein the at least three genes further comprise one or more genes selected from the group consisting of ALK, FGFR2, FGFR3, NTRK1, RET, and ROS1.

6. The method of any one of claims 1-5, wherein the at least three genes further comprise one or more genes selected from the group consisting of APC, AR, ATM, BRAF, BRCA1, BRCA2, CDK4, CDK6, CDK12, EGFR, ERBB2, HRAS, KIT, KRAS, MAPK1, MAPK3, MET, MYC, NRAS, PIK3CA, PTEN, RB1, STK11, and TP53.

7. The method of any one of claims 1-6, wherein the somatic mutation is a substitution, an insertion, a deletion, an amplification, a fusion or a combination thereof.

8. The method of any one of claims 1-7, wherein the somatic mutation is a point mutation, optionally wherein the point mutation is a single-nucleotide variant (SNV) or a multi-nucleotide variant (MNV).

9. The method of any one of claims 1-8, wherein the somatic mutation is an insertion-deletion mutation (indel).

10. The method of any one of claims 1-9, wherein detecting change(s) in the mean level of somatic mutations of the at least three genes in the subject comprises detecting one or more somatic mutations of the at least three genes in the subject (1) during the subject is treated for cancer, (2) before the subject is treated for cancer, (3) after the subject is treated for cancer, or a combination thereof.

11. The method of any one of claims 1-10, wherein detecting change(s) in the mean level of somatic mutations of the at least three genes in the subject comprises detecting one or more somatic mutations of the at least three genes two or more times in the subject, and optionally at least two of the two or more times occur within 5, 7, 14, 28, 35 or 42 days.

12. The method of any one of claims 1-11, wherein change(s) in the mean level of somatic mutations of the at least three genes comprises (1) change(s) in the mean level of somatic mutations of the at least three genes during the subject is treated for cancer, (2) change(s) in the mean level of somatic mutations of the at least three genes from before the subject is treated for cancer to during the subject is treated for cancer, or a combination thereof.

13. The method of any one of claims 1-12, wherein detecting change(s) in the mean level of somatic mutations of at least three genes comprises detecting mean variant allele frequency (VAF) of the at least three genes, optionally wherein the mean variant allele frequency (VAF) is mean mutant allelic frequency (MAF).

14. The method of any one of claims 1-13, wherein change(s) in the mean level of somatic mutations of at least three genes is a ratio of mean MAF of at least one additional time point to a first time point, and wherein the first time point is earlier than the at least one additional time point.

15. The method of any one of claims 1-14, wherein detecting change(s) in the mean level of somatic mutations of the at least three genes in the subject comprises detecting change(s) in the mean level of somatic mutations of the at least three genes in a biological sample from the subject, or derivative thereof.

16. The method of claim 15, wherein the biological sample comprises a bodily fluid, whole blood, plasma, one or more tissues, one or more cells, or a combination thereof.

17. The method of claim 16, wherein the bodily fluid comprises blood, plasma, urine, or a combination thereof, optionally wherein the bodily fluid is blood.

18. The method of any one of claims 15-17, wherein the biological sample comprises circulating tumor DNA (ctDNA), cell-free DNA (cfDNA), circulating tumor cell (CTC), or a combination thereof.

19. The method of claim 18, further comprising analyzing the ctDNA using polymerase chain reaction (PCR) or next generation sequencing (NGS), optionally wherein the NGS is a high-throughput NGS and/or wherein the PCR is digital droplet PCR (ddPCR).

20. The method of any one of claims 1-19, wherein the subject has one or more somatic mutations in the at least three genes before being treated with the PLK1 inhibitor.

21. The method of any one of claims 1-20, wherein detecting change(s) in the mean level of the somatic mutation(s) of the at least three genes in the subject comprises detecting one or more somatic mutations of the at least three genes emerged in the subject after the subject being treated with the PLK1 inhibitor and bevacizumab.

22. The method of any one of claims 1-21, wherein determining the responsiveness of the subject comprises determining if the subject is a responder of the treatment, if the subject is or is going to be in complete recover (CR), or if the subject is or is going to be in partial remission (PR).

23. The method of claim 22, wherein the change in the mean level of the somatic mutation(s) of the at least three genes in the responder is a decrease of at least 50%, at least 75%, at least 90%, at least 95%, or at least 99%, optionally wherein the change in the mean level of the somatic mutations of the at least three genes in the responder is a decrease of at least 99%.

24. The method of claim 22 or 23, wherein the change in the mean level of the somatic mutations of the at least three genes identifies the responders more accurately relative to change(s) in mutation(s) of one gene, optionally wherein the one gene is KRAS.

25. The method of anyone of claims 22-24, wherein a specificity and/or an accuracy of identifying responders using the change in the mean level of the somatic mutations of the at least three genes is at least 3%, at least 5%, at least 7%, at least 10%, at least 15%, or at least 20% higher, relative to using change(s) in mutation(s) of one gene, optionally wherein the one gene is KRAS.

26. The method of any one of claims 1-25, wherein determining the responsiveness of the subject comprises determining progression-free survival (PFS) of the subject.

27. The method of any one of claims 1-26, wherein determining the responsiveness of the subject comprises determining overall survival (OS).

28. The method of any one of claims 1-27, wherein determining the responsiveness of the subject comprises determining if the subject has a partial response to the treatment, if the subject has a complete response to the treatment, if the subject has a stable disease (SD) status, or if the subject has a progressive disease (PD) status.

29. The method of any one of claims 13-28, wherein the cancer treatment with the PLK1 inhibitor and bevacizumab is maintained if the change in mean MAF of the at least three genes is a decrease of at least 25%, at least 50%, or at least 75%, optionally wherein the decrease is at least 99%, and optionally wherein the decrease is detected at the end of cycle 1 of the cancer treatment or at day 1 of cycle 2 of the cancer treatment.

30. The method of any one of claims 13-29, wherein the cancer treatment is for at least one week, at least two weeks, at least three weeks, at least one month, at least three months, or at least six months.

31. The method of any one of claims 13-30, wherein the cancer treatment with the PLK1 inhibitor and bevacizumab is modified or discontinued if the change in mean MAF the at least three genes is a decrease of less than 50%, less than 25%, or less than 10%, optionally wherein the decrease is detected at the end of cycle 1 of the cancer treatment or at day 1 of cycle 2 of the cancer treatment.

32. The method of any one of claims 1-31, wherein the cancer treatment with the PLK1 inhibitor and bevacizumab is maintained if mean level of mutations in the at least three genes in the samples decreases to below 0.01% or below 0.001% of mean level of the at least three genes in the sample.

33. A method of improving outcome of a treatment for colorectal cancer, comprising:
detecting mean variant allele frequency of a first set of at least three genes in a subject at a first time point in a first sample, wherein the first set of the at least three genes comprise KRAS, APC and TP53 genes, wherein the first time point is before the subject starts the treatment for colorectal cancer, or during the treatment for colorectal cancer, and wherein the treatment for colorectal cancer comprises administering a PLK1 inhibitor and bevacizumab to the subject;
detecting mean variant allele frequency of a second set of at least three gene in the subject at one or more additional time points in one or more additional samples, wherein the second set of the at least three genes comprise KRAS, APC and TP53 genes, and wherein the at least one of the one or more additional time points is during the treatment for colorectal cancer;
determining the difference of the mean variant allele frequency between the first and the one or more additional samples, wherein a decrease in the mean variant allele frequency in at least one of the one or more additional samples relative to the first sample indicates the subject as responsive to the treatment for colorectal cancer; and
continuing the treatment for colorectal cancer to the subject if the subject is indicated as responsive to the treatment for colorectal cancer, or discontinuing the treatment for colorectal cancer to the subject and/or starting a different treatment to the subject if the subject is not indicated as responsive to the treatment for colorectal cancer.

34. The method of claim 33, wherein the first time point is before the subject starts the treatment for colorectal cancer.

35. The method of any one of claims 33-34, wherein at least two of the additional time points are during the treatment for colorectal cancer.

36. A method of treating colorectal cancer, comprising:
treating a subject with colorectal cancer, wherein the treating comprises administering a PLK1 inhibitor and bevacizumab to the subject;
determining a decrease, relative to a mean variant allele frequency of a first set of at least three genes in a first sample of the subject obtained at a first time point before the subject receives the treatment for colorectal cancer or during the treatment for colorectal cancer, in a mean variant allele frequency of a second set of at least three gene in a second sample of the subject obtained at a second time point after the subject starts receiving the treatment for colorectal cancer; and
continuing with the treatment for colorectal cancer,
wherein the first set of at least three genes and the second set of at least three genes comprise KRAS, APC and TP53 genes.

37. The method of any one of claims 33-36, wherein the first set of at least three genes detected at the first time point are the same as the second set of at least three genes detected at the second time point.

38. The method of any one of claims 33-36, wherein the first set of at least three genes detected at the first time point are different from the second set of at least three genes detected at the second time point.

39. The method of any one of claims 33-38, wherein the first set of at least three genes and/or the second set of at least three genes further comprise one or more genes selected from the group consisting of AKT1, ALK, AR, ARAF, AR/01A, ATM, BRAF, BRCA1, BRCA2, CCND1, CCND2, CCNE1, COH1, CDK4, CDK6, CDK12, COKN2A, CTNNB1, DDR2, EGFR, ERBB2, ESR1, EZH2, FBXW1, FGFR1, FGFR2, FGFR3, G4TA3, GNA11, GNAQ, GNAS, HNF1A, HRAS, IDH1, IDH2, JAK2, JAK3, KIT, MAP2K1, MAP2K2, MAPK1, MAPK3, MET, MLH1, MPL, MTOR, MYC, NF1, NFE2L2, NOTCH1, NPM1, NRAS, NTRK1, NTRK3, POGFRA, PIK3CA, PTEN, PTPN11, RAF1, RB1, RET, RHEB, RHOA, RIT1, ROS1, SMA04, SMO, STK11, TERT, TSC1, and VHL.

40. The method of any one of claims 33-39, wherein the first set of at least three genes and/or the second set of at least three genes further comprise one or more genes selected from the group consisting of AR, BRAF, CCND1, CCND2, CCNE1, COK4, COK6, EGFR, ERBB2, FGFR1, FGFR2, KIT, MET, MYC, POGFRA, PIK3CA, and RAF1.

41. The method of any one of claims 33-40, wherein the first set of at least three genes and/or the second set of at least three genes further comprise one or more genes selected from the group consisting of ALK, FGFR2, FGFR3, NTRK1, RET, and ROS1.

42. The method of any one of claims 33-41, wherein the first set of at least three genes and/or the second set of at least three genes further comprise one or more genes selected from the group consisting of APC, AR, ATM, BRAF, BRCA1, BRCA2, CDK4, CDK6, CDK12, EGFR, ERBB2, HRAS, KIT, KRAS, MAPK1, MAPK3, MET, MYC, NRAS, PIK3CA, PTEN, RB1, STK11, and TP53.

43. The method of any one of claims 33-42, wherein the first set of at least three genes and/or the second set of at least three genes have somatic mutation(s); and optionally wherein the somatic mutation is a substitution, an insertion, a deletion, an amplification, a fusion or a combination thereof, optionally wherein the somatic mutation is a point mutation and/or an insertion-deletion mutation (indel), further optionally wherein the point mutation is a single-nucleotide variant (SNV).

44. The method of claim 43, wherein the somatic mutation(s) in the first set of at least three genes detected at the first time point are the same as the somatic mutation(s) in the second set of at least three genes detected at the second time point.

45. The method of claim 43, wherein the somatic mutation(s) in the first set of at least three genes detected at the first time point are different from the somatic mutation(s) in the second set of at least three genes detected at the second time point.

46. The method of any one of claims 33-45, wherein the first time point is prior or immediately prior to the cancer treatment.

47. The method of any one of claims 33-45, wherein the first time point is during the cancer treatment, and optionally at day 5, 7, 14, or 28 of the cancer treatment.

48. The method of any one of claims 33-47, wherein the one or more additional time points are during the cancer treatment, and optionally on day 5, 7, 14, 28, 35 or 42 of the cancer treatment.

49. The method of any one of claims 33-48, wherein the first time point and at least one of the one or more additional time points are during the first cycle of the cancer treatment.

50. The method of any one of claims 33-49, wherein at least one of the one or more additional time points are during the first cycle of the cancer treatment, and at least one of the one or more additional time points are during the second cycle of the cancer treatment.

51. The method of any one of claims 33-50, wherein the mean variant allele frequency is mean mutant allelic frequency (MAF).

52. The method of any one of claims 33-51, wherein the detecting step comprises detecting mean variant allele frequency in the first set of at least three genes and/or the second set of at least three genes in a biological sample from the subject, or derivative thereof; optionally wherein the biological sample comprises a bodily fluid, whole blood, plasma, one or more tissues, one or more cells, or a combination thereof, further optionally wherein the bodily fluid comprises blood, plasma, urine, or a combination thereof.

53. The method of claim 52, wherein the biological sample comprises circulating tumor DNA (ctDNA), circulating tumor cell (CTC), or a combination thereof.

54. The method of claim 53, comprising analyzing the ctDNA using polymerase chain reaction (PCR) or next generation sequencing (NGS), and optionally wherein the NGS is a high-throughput NGS.

55. The method of any one of claims 33-54, wherein the subject has one or more mutations in the first set of at least three genes and/or the second set of at least three genes before being treated with the PLK1 inhibitor and bevacizumab.

56. The method of any one of claims 33-54, wherein the subject does not have mutations in the first set of at least three genes and/or the second set of at least three genes before being treated with the PLK1 inhibitor and bevacizumab.

57. The method of any one of claims 33-56, wherein the mean variant allele frequency of a second set of at least three gene decreases to below 0.01% or below 0.001% of mean level of the at least three genes in a sample.

58. The method of any one of claims 1-57, wherein the subject has received one or more prior cancer treatment.

59. The method of any one of claims 1-58, wherein the colorectal cancer is metastatic colorectal cancer.

60. The method of any one of claims 1-59, wherein the PLK1 inhibitor is onvansertib, BI2536, volasertib (BI 6727), GSK461364, HMN-176, HMN-214, AZD1775, CYC140, rigosertib (ON-01910), MLN0905, TKM-080301, TAK-960, Ro3280, or a combination thereof, optionally wherein the PLK1 inhibitor is onvansertib.

61. The method of claim 60, wherein the treatment for colorectal cancer comprises administration of onvansertib every day in a cycle of 28 days.

62. The method of claim 61, wherein the treatment for colorectal cancer comprises administration of onvansertib for the first 21 days and not the last 7 days in a cycle of 28 days.

63. The method of claim 61, wherein the treatment for colorectal cancer comprises administration of onvansertib for ten days in a cycle of 28 days.

64. The method of claim 61, wherein the treatment for colorectal cancer comprises administration of onvansertib for five days in the first 14 days and five days in the second 14 days in a cycle of 28 days, optionally wherein the treatment for colorectal cancer comprises administration of onvansertib on days 1-5 and days 15-19 in a cycle of 28 days.

65. The method of any one of claims 61-64, wherein the treatment comprises administration of onvansertib at 6 mg/m²-24 mg/m², optionally 6 mg/m²-12 mg/m² or 12 mg/m²-18 mg/m², further optionally 12 mg/m² or 15 mg/m².

66. The method of any one of claims 61-65, wherein a maximum concentration (Cₘₐₓ) of onvansertib in a blood of the subject is from about 100 nmol/L to about 1500 nmol/L.

67. The method of any one of claims 61-66, wherein an area under curve (AUC) of a plot of a concentration of onvansertib in a blood of the subject over time is from about 1000 nmol/L·hour to about 400000 nmol/L·hour.

68. The method of any one of claims 61-67, wherein a time (Tmax) to reach a maximum concentration of onvansertib in a blood of the subject is from about 1 hour to about 5 hours.

69. The method of any one of claims 61-68, wherein an elimination half-life (T_{1/2}) of onvansertib in a blood of the subject is from about 10 hours to about 60 hours.

70. The method of any one of claims 1-69, wherein the treatment for colorectal cancer further comprises administering to the subject at least one additional cancer therapeutics or cancer therapy; optionally wherein the PLK inhibitor and the additional cancer therapeutics or cancer therapy are co-administered simultaneously or sequentially.

71. The method of any one of claims 1-70, wherein the cancer treatment comprises one or more cycles, and change(s) in the mean level of somatic mutations of the at least three genes or mean variant allele frequency of the first set of at least three genes and/or the second set of at least three genes is detected before, during and/or after each cycle of the cancer treatment.

72. The method of claim 71, wherein each cycle of treatment is at least 21 days.

73. The method of claim 71, wherein each cycle of treatment is from about 21 days to about 28 days, optionally 28 days.

74. The method of any one of claims 1-73, wherein the subject is human.

75. The method of any one of claims 2-74, wherein the chemotherapy is FOLFIRI, FOLFOX, XELOX (CAPOX), or FOLFOXIRI.

76. Use of a PLK1 inhibitor and bevacizumab as a treatment of a subject with colorectal cancer, wherein the responsiveness of the subject to the treatment for colorectal cancer is determined using a method of any one of claims 1-32.

77. Use of a PLK1 inhibitor and bevacizumab as a treatment of a subject with colorectal cancer, wherein the treatment outcome is improved using a method of any one of claims 33-75.

78. Use of a PLK1 inhibitor and bevacizumab as a treatment of a subject with colorectal cancer, wherein the subject is treated using a method of any one of claims 33-75.

79. The use of any one of claims 76-78, wherein the PLK1 inhibitor is onvansertib.
